(19) United States
(12) Patent Application Publication
(10) Pub. No.: US 2004/0235667 A1
(43) Pub. Date: Nov. 25, 2004

(54) Pyrazolyl-substituted thienyloxypyridines

(76) Inventors: Michael Hoffmann, Ludwigshafen (DE); Liliana Parra Rapado, Mannheim (DE); Wolfgang von Deyn, Neustadt (DE); Ernst Baumann, Dudenhofen (DE); Markus Kordes, Frankenthal (DE); Ulf Misslitz, Neustadt (DE); Matthias Witschel, Bad Durkheim (DE); Cyrill Zagar, Mannheim (DE); Andreas Landes, Romerberg Heiligenstein (DE)

Correspondence Address: KEIL & WEINKAUF, 1350 CONNECTICUT AVENUE, N.W., WASHINGTON, DC, 20036, US

(21) Appl. No.: 10/487,453

(22) PCT Filed: Aug. 31, 2002
(86) PCT No.: PCT/EP02/09750

(30) Foreign Application Priority Data
Sep. 7, 2001 (DE) 10144185.1

(51) Int. Cl. A01N 43/48; C 07D 4 9/14

(52) U.S. Cl. 504/253; 546/275.4

(57) ABSTRACT

Pyrazolyl-substituted thienyloxypyridines of the formula I

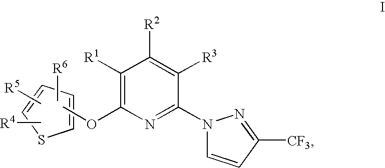

where:

R¹, R³ are hydrogen, halogen, cyano, nitro, alkyl, haloalkyl, alkoxy or haloalkoxy;

R² is hydrogen, halogen, cyano, alkenyl, alkynyl, haloalkyl, haloalkenyl, haloalkynyl, alkoxy, alkenyloxy, alkynyloxy, haloalkoxy, alkoxyalkyl, alkylamino, di(alkyl)amino, alkylthio, haloalkylthio, alkylsulfinyl, haloalkylsulfinyl, alkylsulfonyl, haloalkylsulfonyl or COR⁷;

R⁴, R⁵, R⁶ are hydrogen, halogen, cyano, alkyl, haloalkyl, alkoxy, haloalkoxy, alkylthio, haloalkylthio, alkylsulfonyl or haloalkylsulfonyl;

R⁷ is hydrogen, hydroxyl, alkyl, alkoxy, amino, alkylamino or di(alkyl)amino;

and their agriculturally useful salts;

processes and intermediates for their preparation; and the use of these compounds or of the compositions comprising them for controlling unwanted plants are described.

The present invention relates to pyrazolyl-substituted thienyloxypyridines of the formula I

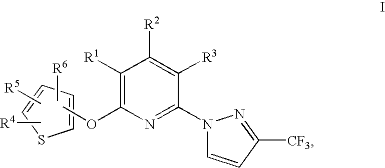

where

R¹, R³ are hydrogen, halogen, cyano, nitro, C₁-C₆-alkyl, C₁-C₆-haloalkyl, C₁-C₆-alkoxy or C₁-C₆-haloalkoxy;

R² is hydrogen, halogen, cyano, C₂-C₆-alkenyl, C₂-C₆-alkynyl, C₁-C₆-haloalkyl, C₂-C₆-haloalkenyl, C₂-C₆-haloalkynyl, C₁-C₆-alkoxy, C₃-C₆-alkenyloxy, C₃-C₆-alkynyloxy, C₁-C₆-haloalkoxy, C₁-C₆-alkoxy-C₁-C₄-alkyl, C₁-C₆-alkylamino, di(C₁-C₄-alkyl)amino, C₁-C₆-alkylthio, C₁-C₆-haloalkylthio, C₁-C₆-alkylsulfinyl, C₁-C₆-haloalkylsulfinyl, C₁-C₆-alkylsulfonyl, C₁-C₆-haloalkylsulfonyl or COR⁷;

R⁴, R⁵, R⁶ are hydrogen, halogen, cyano, C₁-C₆-alkyl, C₁-C₆-haloalkyl, C₁-C₆-alkoxy, C₁-C₆-haloalkoxy, C₁-C₆-alkylthio, C₁-C₆-haloalkylthio, C₁-C₆-alkylsulfonyl or C₁-C₆-haloalkylsulfonyl;

R⁷ is hydrogen, hydroxyl, C₁-C₆-alkyl, C₁-C₆-alkoxy, amino, C₁-C₆-alkylamino or di(C₁-C₄-alkyl)amino;

and their agriculturally useful salts.

Moreover, the invention relates to intermediates and processes for preparing compounds of the formula I, to compositions comprising them and to the use of these derivatives or of the compositions comprising these derivatives for controlling harmful plants.

WO 99/24427 and EP-A-1 101 764 disclose herbidically active thienyloxyazines and 2-aryloxy-6-pyrazole pyridines.

However, the herbicidal properties of the prior-art compounds and/or their compatibility with crop plants are not entirely satisfactory.

It is an object of the present invention to provide in particular herbicidally active compounds having improved properties.

We have found that this object is achieved by the pyrazolyl-substituted thienyloxypyridines of the formula I and their herbidical action.

Furthermore, we have found herbicidal compositions which comprise the compounds I and have very good herbicidal action. Moreover, we have found processes for preparing these compositions and methods for controlling undesirable vegetation using the compounds I.

Depending on the substitition pattern, the compounds of the formula I may contain one or more centers of chirality, in which case they are present as enantiomers or mixtures of diastereomers. The invention provides both the pure enantiomers or diastereomers and their mixtures.

The compounds of the formula I can also be present in the form of their agriculturally useful salts, the type of salt generally being immaterial. Suitable are, in general, the salts of those cations and the acid addition salts of those acids whose cations and anions, respectively, do not adversely affect the herbicidal action of the compounds I.

Suitable cations are in particular ions of the alkali metals, preferably lithium, sodium and potassium, of the alkaline earth metals, preferably calcium and magnesium, and of the transition metals, preferably manganese, copper, zinc and iron, and also ammonium, where, if desired, 1 to 4 hydrogen atoms may be replaced by C₁-C₄-alkyl, hydroxy-C₁-C₄-alkyl, C₁-C₄-alkoxy-C₁-C₄-alkyl, hydroxy-C₁-C₄-alkoxy-C₁-C₄-alkyl, phenyl or benzyl, preferably ammonium, dimethylammonium, diisopropylammonium, tetramethylammonium, tetrabutylammonium, 2-(2-hydroxyethloxy)eth-1-ylammonium, di(2-hydroxyeth-1-yl)ammonium, trimethylbenzylammonium, furthermore phosphonium ions, sulfonium ions, preferably tri(C₁-C₄-alkyl)sulfonium, and sulfoxonium ions, preferably tri(C₁-C₄-alkyl)sulfoxonium.

Anions of useful acid addition salts are preferably chloride, bromide, fluoride, hydrogensulfate, sulfate, dihydrogenphosphate, hydrogenphosphate, nitrate, bicarbonate, carbonate, hexafluorosilicate, hexafluorophosphate, benzoate, and also the anions of C₁-C₄-alkanoic acids, preferably formate, acetate, propionate and butyrate.

The organic moieties mentioned for the substituents R¹-R⁷ are collective terms for individual enumerations of the individual group members. All hydrocarbon chains, i.e. all alkyl, alkenyl, alkynyl, haloalkyl, haloalkenyl, haloalkynyl, alkoxy, alkenyloxy, alkynyloxy, haloalkoxy, alkoxyalkyl, alkylamino, dialkylamino, alkylthio, haloalkylthio, alkylsulfinyl, haloalkylsulfinyl, alkylsulfonyl and haloalkylsulfonyl moieties can be straight-chain or branched. Unless indicated otherwise, halogenated substituents preferably carry one to five, in particular one to three, identical or different halogen atoms. The term ‘halogen’ denotes in each case fluorine, chlorine, bromine or iodine.

Examples of other meanings are:

C₁-C₄-alkyl: and the alkyl moieties of hydroxy-C₁-C₄-alkyl, hydroxy-C₁-C₄-alkoxy-C₁-C₄-alkyl, tri(C₁-C₄-alkyl)sulfonium and tri(C₁-C₄-alkyl)sulfoxonium: for example methyl, ethyl, 1-propyl, 1-methylethyl, 1-butyl, 1-methylpropyl, 2-methylpropyl and 1,1-dimethylethyl;

C₁-C₆-alkyl: C₁-C₄-alkyl as mentioned above, and also, for example, 1-pentyl, 1-methylbutyl, 2-methylbutyl, 3-methylbutyl, 2,2-dimethylpropyl, 1-ethylpropyl, 1-hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1-ethyl-1-methylpropyl and 1-ethyl-3-methylpropyl;

C₂-C₆-alkenyl: for example ethenyl, 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl, 2-methyl-2-propenyl, 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl and 1-ethyl-2-methyl-2-propenyl;

C₂-C₆-alkynyl: for example ethynyl, 1-propynyl, 2-propynyl, 1-butynyl, 2-butynyl, 3-butynyl, 1-methyl-2-propynyl, 1-pentynyl, 2-pentynyl, 3-pentynyl, 4-pentynyl, 1-methyl-2-butynyl, 1-methyl-3-butynyl, 2-methyl-3-butynyl, 3-methyl-1-butynyl, 1,1-dimethyl-2-propynyl, 1-ethyl-2-propynyl, 1-hexynyl, 2-hexynyl, 3-hexynyl, 4-hexynyl, 5-hexynyl, 1-methyl-2-pentynyl, 1-methyl-3-pentynyl, 1-methyl-4-pentynyl, 2-methyl-3-pentynyl, 2-methyl-4-pentynyl, 3-methyl-1-pentynyl, 3-methyl-4-pentynyl, 4-methyl-1-pentynyl, 4-methyl-2-pentynyl, 1,1-dimethyl-2-butynyl, 1,1-dimethyl-3-butynyl, 1,2-dimethyl-3-butynyl, 2,2-dimethyl-3-butynyl, 3,3-dimethyl-1-butynyl, 1-ethyl-2-butynyl, 1-ethyl-3-butynyl, 2-ethyl-3-butynyl and 1-ethyl-1-methyl-2-propynyl;

C₁-C₆-haloalkyl: a C₁-C₆-alkyl radical as mentioned above which is partially or fully substituted by fluorine, chlorine, bromine and/or iodine, i.e., for example, chloromethyl, dichloromethyl, trichloromethyl, fluoromethyl, difluoromethyl, trifluoromethyl, chlorofluoromethyl, dichlorofluoromethyl, chlorodifluoromethyl, 2-fluoroethyl, 2-chloroethyl, 2-bromoethyl, 2-iodoethyl, 2,2-difluoroethyl, 2,2,2-trifluoroethyl, 2-chloro-2-fluoroethyl, 2-chloro-2,2-difluoroethyl, 2,2-dichloro-2-fluoroethyl, 2,2,2-trichloroethyl, pentafluoroethyl, 2-fluoropropyl, 3-fluoropropyl, 2,2-difluoropropyl, 2,3-difluoropropyl, 2-chloropropyl, 3-chloropropyl, 2,3-dichloropropyl, 2-bromopropyl, 3-bromopropyl, 3,3,3-trifluoropropyl, 3,3,3-trichloropropyl, 2,2,3,3,3-pentafluoropropyl, heptafluoropropyl, 1-(fluoromethyl)-2-fluoroethyl, 1-(chloromethyl)-2-chloroethyl, 1-(bromomethyl)-2-bromoethyl, 4-fluorobutyl, 4-chlorobutyl, 4-bromobutyl, nonafluorobutyl, 5-fluoropentyl, 5-chloropentyl, 5-bromopentyl, 5-iodopentyl, undecafluoropentyl, 6-fluorohexyl, 6-chlorohexyl, 6-bromohexyl, 6-iodohexyl and dodecafluorohexyl;

C₂-C₆-haloalkenyl: a C₂-C₆-alkenyl radical as mentioned above which is partially or fully substituted by fluorine, chlorine, bromine and/or iodine, for example 2-chlorovinyl, 2-chloroallyl, 3-chloroallyl, 2,3-dichloroallyl, 3,3-dichloroallyl, 2,3,3-trichloroallyl, 2,3-dichlorobut-2-enyl, 2-bromovinyl, 2-bromoallyl, 3-bromoallyl, 2,3-dibromoallyl, 3,3-dibromoallyl, 2,3,3-tribromoallyl or 2,3-dibrombut-2-enyl;

C₂-C₆-haloalkynyl: a C₂-C₆-alkynyl radical as mentioned above which is partially or fully substituted by fluorine, chlorine, bromine and/or iodine, for example 1,1-difluoroprop-2-yn-1-yl, 3-iodoprop-2-yn-1-yl, 4-fluorobut-2-yn-1-yl, 4-chlorobut-2-yn-1-yl, 1,1-difluorobut-2-yn-1-yl, 4-iodobut-3-yn-1-yl, 5-fluoropent-3-yn-1-yl, 5-iodopent-4-yn-1-yl, 6-fluorohex-4-yn-1-yl or 6-iodohex-5-yn-1-yl;

C₁-C₄-alkoxy and the alkoxy moieties of hydroxy-C₁-C₄-alkoxy-C₁-C₄-alkyl: for example methoxy, ethoxy, propoxy, 1-methylethoxy, butoxy, 1-methylpropoxy, 2-methylpropoxy and 1,1-dimethylethoxy;

C₁-C₆-alkoxy: C₁-C₄-alkoxy as mentioned above and also, for example, pentoxy, 1-methylbutoxy, 2-methylbutoxy, 3-methoxylbutoxy, 1,1-dimethylpropoxy, 1,2-dimethylpropoxy, 2,2-dimethylpropoxy, 1-ethylpropoxy, hexoxy, 1-methylpentoxy, 2-methylpentoxy, 3-methylpentoxy, 4-methylpentoxy, 1,1-dimethylbutoxy, 1,2-dimethylbutoxy, 1,3-dimethylbutoxy, 2,2-dimethylbutoxy, 2,3-dimethylbutoxy, 3,3-dimethylbutoxy, 1-ethylbutoxy, 2-ethylbutoxy, 1,1,2-trimethylpropoxy, 1,2,2-trimethylpropoxy, 1-ethyl-1-methylpropoxy and 1-ethyl-2-methylpropoxy;

C₃-C₆-alkenyloxy: for example prop-1-en-1-yloxy, prop-2-en-1-yloxy, 1-methylethenyloxy, buten-1-yloxy, buten-2-yloxy, buten-3-yloxy, 1-methyl-prop-1-en-1-yloxy, 2-methylprop-1-en-1-yloxy, 1-methylprop-2-en-1-yloxy, 2-methylprop-2-en-1-yloxy, penten-1-yloxy, penten-2-yloxy, penten-3-yloxy, penten-4-yloxy, 1-methylbut-1-en-1-yloxy, 2-methylbut-1-en-1-yloxy, 3-methylbut-1-en-1-yloxy, 1-methylbut-2-en-1-yloxy, 2-methylbut-2-en-1-yloxy, 3-methylbut-2-en-1-yloxy, 1-methylbut-3-en-1-yloxy, 2-methylbut-3-en-1-yloxy, 3-methylbut-3-en-1-yloxy, 1,1-dimethylprop-2-en-1-yloxy, 1,2-dimethylprop-1-en-1-yloxy , 1,2-dimethylprop-2-en-1-yloxy, 1-ethylprop-1-en-2-yloxy, 1-ethylprop-2-en-1-yloxy, hex-1-en-1-yloxy, hex-2-en-1-yloxy, hex-3-en-1-yloxy, hex-4-en-1-yloxy, hex-5-en-1-yloxy, 1-methylpent-1-en-1-yloxy, 2-methylpent-1-en-1-yloxy, 3-methylpent-2-en-1-yloxy, 4-methylpent-2-en-1-yloxy, 1-methylpent-2-en-1-yloxy, 2-methylpent-2-en-1-yloxy, 3-methylpent-2-en-1-yloxy, 4-methylpent-2-en-1-yloxy, 1-methylpent-3-en-1-yloxy, 2-methylpent-3-en-1-yloxy, 3-methylpent-3-en-1-yloxy, 4-methylpent-3-en-1-yloxy, 1-methylpent-4-en-1-yloxy, 2-methylpent-4-en-1-yloxy, 3-methylpent-4-en-1-yloxy, 4-methylpent-4-en-1-yloxy, 1,1-dimethylbut-2-en-1-yloxy, 1,2-dimethylbut-3-en-1-yloxy, 1,2-dimethylbut-3-en-1-yloxy, 1,2-dimethylbut-2-en-1-yloxy, 1,2-dimethylbut-3-en-1-yloxy, 1,3-dimethylbut-3-en-1-yloxy, 1,3-dimethylbut-2-en-1-yloxy, 1,3-dimethylbut-3-en-1-yloxy, 2,2-dimethylbut-3-en-1-yloxy, 2,3-dimethylbut-3-en-1-yloxy, 2,3-dimethylbut-2-en-1-yloxy, 2,3-dimethylbut-3-en-1-yloxy, 3,3-dimethylbut-1-en-1-yloxy, 3,3-dimethylbut-2-en-1-yloxy , 1-ethylbut-3-en-1-yloxy, 2-ethylbut-2-en-1-yloxy, 1-ethylbut-3-en-1-yloxy, 2-ethylbut-3-en-1-yloxy, 2-ethylbut-2-en-1-yloxy, 2-ethylbut-3-en-1-yloxy, 1,1,2-trimethylprop-2-en-1-yloxy, 1-ethyl-1-methyl-prop-2-en-1-yloxy, 1-ethyl-2-methylprop-1-en-1-yloxy and 1-ethyl-2-methylprop-2-en-1-yloxy;

C₃-C₆-alkynyloxy: for example prop-1-yn-1-yloxy, prop-2-yn-1-yloxy, but-1-yn-1-yloxy, but-1-yn-3-yloxy, but-1-yn-4-yloxy, but-2-yn-1-yloxy, pent-1-yn-1-yloxy, pent-1-yn-3-yloxy, pent-1-yn-4-yloxy, pent-1-yn-5-yloxy, pent-2-yn-1-yloxy, pent-2-yn-4-yloxy, pent-2-yn-5-yloxy, 3-methylbut-1-yn-3-yloxy, 3-methylbut-1-yn-4-yloxy, hex-1-yn-1-yloxy, hex-1-yn-3-yloxy, hex-1-yn-4-yloxy, hex-1-yn-5-yloxy, hex-1-yn-6-yloxy, hex-2-yn-1-yloxy, hex-2-yn-4-yloxy, hex-2-yn-5-yloxy, hex-2-yn-6-yloxy, hex-3-yn-1-yloxy, hex-3-yn-2-yloxy, 3-methylpent-1-yn-1-yloxy, 3-methylpent-1-yn-3-yloxy, 3-methylpent-1-yn-4-yloxy, 3-methylpent-1-yn-5-yloxy, 4-methylpent-1-yn-1-yloxy, 4-methylpent-2-yn-4-yloxy and 4-methylpent-2-yn-5-yloxy;

C₁-C₆-haloalkoxy: a C₁-C₆-alkoxy radical as mentioned above which is partially or fully substituted by fluorine, chlorine, bromine and/or iodine, i.e., for example, fluoromethoxy, difluoromethoxy, trifluoromethoxy, chlorodifluoromethoxy, bromodifluoromethoxy, 2-fluoroethoxy, 2-chloroethoxy, 2-bromomethoxy, 2-iodoethoxy, 2,2-difluoroethoxy, 2,2,2-trifluoroethoxy, 2-chloro-2-fluoroethoxy, 2-chloro-2,2-difluoroethoxy, 2,2-dichloro-2-fluoroethoxy, 2,2,2-trichloroethoxy, pentafluoroethoxy, 2-fluoropropoxy, 3-fluoropropoxy, 2-chloropropoxy, 3-chloropropoxy, 2-bromopropoxy, 3-bromopropoxy, 2,2-difluoropropoxy, 2,3-difluoropropoxy, 2,3-dichloropropoxy, 3,3,3-trifluoropropoxy, 3,3,3-trichloropropoxy, 2,2,3,3,3-pentafluoropropoxy, heptafluoropropoxy, 1-(fluoromethyl)-2-fluoroethoxy, 1-(chloromethyl)-2-chloroethoxy, 1-(bromomethyl)-2-bromoethoxy, 4-fluorobutoxy, 4-chlorobutoxy, 4-bromobutoxy, nonafluorobutoxy, 5-fluoropentoxy, 5-chloropentoxy, 5-bromopentoxy, 5-iodopentoxy, undecafluoropentoxy, 6-fluorohexoxy, 6-chlorohexoxy, 6-bromohexoxy, 6-iodohexoxy and dodecafluorohexoxy;

C₁-C₆-alkoxy-C₁-C₄-alkyl: C₁-C₄-alkyl which is substituted by C₁-C₆-alkoxy as mentioned above, i.e., for example, methoxymethyl, ethoxymethyl, propoxymethyl, (1-methylethoxy)methyl, butoxymethyl, (1-methylpropoxy)methyl, (2-methylpropoxy)methyl, (1,1-dimethylethoxy)methyl, 2-(methoxy)ethyl, 2-(ethoxy)ethyl, 2-(propoxy)ethyl, 2-(1-methylethoxy)ethyl, 2-(butoxy)ethyl, 2-(1-methylpropoxy)ethyl, 2-(2-methylpropoxy)ethyl, 2-(1,1-dimethylethoxy)ethyl, 2-(methoxy)propyl, 2-(ethoxy)propyl, 2-(propoxy)propyl, 2-(1-methylethoxy)propyl, 2-(butoxy)propyl, 2-(1-methylpropoxy)propyl, 2-(2-methylpropoxy)propyl, 2-(1,1-dimethylethoxy)propyl, 3-(methoxy)propyl, 3-(ethoxy)propyl, 3-(propoxy)propyl, 3-(1-methylethoxy)propyl, 3-(butoxy)propyl, 3-(1-methylpropoxy)propyl, 3-(2-methylpropoxy)propyl, 3-(1,1-dimethylethoxy)propyl, 2-(methoxy)butyl, 2-(ethoxy)butyl, 2-(propoxy)butyl, 2-(1-methylethoxy)butyl, 2-(butoxy)butyl, 2-(1-methylpropoxy)butyl, 2-(2-methylpropoxy)butyl, 2-(1,1-dimethylethoxy)butyl, 3-(methoxy)butyl, 3-(ethoxy)butyl, 3-(propoxy)butyl, 3-(1-methylethoxy)butyl, 3-(butoxy)butyl, 3-(1-methylpropoxy)butyl, 3-(2-methylpropoxy)butyl, 3-(1,1-dimethylethoxy)butyl, 4-(methoxy)butyl, 4-(ethoxy)butyl, 4-(propoxy)butyl, 4-(1-methylethoxy)butyl, 4-(butoxy)butyl, 4-(1-methylpropoxy)butyl, 4-(2-methylpropoxy)butyl and 4-(1,1-dimethylethoxy)butyl;

C₁-C₆-alkylamino: for example methylamino, ethylamino, propylamino, 1-methylethylamino, butylamino, 1-methylpropylamino, 2-methylpropylamino, 1,1-dimethylethylamino, pentylamino, 1-methylbutylamino, 2-methylbutylamino, 3-methylbutylamino, 2,2-dimethylpropylamino, 1-ethylpropylamino, hexylamino, 1,1-dimethylpropylamino, 1,2-dimethylpropylamino, 1-methylpentylamino, 2-methylpentylamino, 3-methylpentylamino, 4-methylpentylamino, 1,1-dimethylbutylamino, 1,2-dimethylbutylamino, 1,3-dimethylbutylamino, 10 2,2-dimethylbutylamino, 2,3-dimethylbutylamino, 3,3-dimethylbutylamino, 1-ethylbutylamino, 2-ethylbutylamino, 1,1,2-trimethylpropylamino, 1,2,2-trimethylpropylamino, 1-ethyl-1-methylpropylamino or 1-ethyl-2-methylpropylamino;

di-(C₁-C₄-alkyl)-amino: for example N,N-dimethylamino, N,N-diethylamino, N,N-dipropylamino, N,N-di(1-methylethyl)amino, N,N-dibutylamino, N,N-di(1-methylpropyl)amino, N,N-di(2-methylpropyl)amino, N,N-di(1,1-dimethylethyl)amino, N-ethyl-N-methylamino, N-methyl-N-propylamino, N-methyl-N-(1-methylethyl)amino, N-butyl-N-methylamino, N-methyl-N-(1-methylpropyl)amino, N-methyl-N-(2-methylpropyl)amino, N-(1,1-dimethylethyl)-N-methylamino, N-ethyl-N-propylamino, N-ethyl-N-(1-methylethyl)amino, N-butyl-N-ethylamino, N-ethyl-N-(1-methylpropyl)amino, N-ethyl-N-(2-methylpropyl)amino, N-ethyl-N-(1,1-dimethylethyl)amino, N-(1-methylethyl)-N-propylamino, N-butyl-N-propylamino, N-(1-methylpropyl)-N-propylamino, N-(2-methylpropyl)-N-propylamino, N-(1,1-dimethylethyl)-N-propylamino, N-butyl-N-(1-methylethyl)amino, N-(1-methylethyl)-N-(1-methylpropyl)amino, N-(1-methylethyl)-N-(2-methylpropyl)amino, N-(1,1-dimethylethyl)-N-(1-methylethyl)amino, N-butyl-N-(1-methylpropyl)amino, N-butyl-N-(2-methylpropyl)amino, N-butyl-N-(1,1-dimethylethyl)amino, N-(1-methylpropyl)-N-(2-methylpropyl)amino, N-(1,1-dimethylethyl)-N-(1-methylpropyl)amino and N-(1,1-dimethylethyl)-N-(2-methylpropyl)amino;

C₁-C₆-alkylthio: for example methylthio, ethylthio, propylthio, 1-methylethylthio, butylthio, 1-methylpropylthio, 2-methylpropylthio and 1,1-dimethylethylthio, pentylthio, 1-methylbutylthio, 2-methylbutylthio, 3-methylbutylthio, 2,2-dimethylpropylthio, 1-ethylpropylthio, hexylthio, 1,1-dimethylpropylthio, 1,2-dimethylpropylthio, 1-methylpentylthio, 2-methylpentylthio, 3-methylpentylthio, 4-methylpentylthio, 1,1-dimethylbutylthio, 1,2-dimethylbutylthio, 1,3-dimethylbutylthio, 2,2-dimethylbutylthio, 2,3-dimethylbutylthio, 3,3-dimethylbutylthio, 1-ethylbutylthio, 2-ethylbutylthio, 1,1,2-trimethylpropylthio, 1,2,2-trimethylpropylthio, 1-ethyl-1-methylpropylthio and 1-ethyl-2-methylpropylthio;

C₁-C₆-haloalkylthio: a C₁-C₆-alkylthio radical as mentioned above which is partially or fully substituted by fluorine, chlorine, bromine and/or iodine, i.e., for example, fluoromethylthio, difluoromethylthio, trifluoromethylthio, chlorodifluoromethylthio, bromodifluoromethylthio, 2-fluoroethylthio, 2-chloroethylthio, 2-bromoethylthio, 2-iodoethylthio, 2,2-difluoroethylthio, 2,2,2-trifluoroethylthio, 2,2,2-trichloroethylthio, 2-chloro-2-fluoroethylthio, 2-chloro-2,2-difluoroethylthio, 2,2-dichloro-2-fluoroethylthio, pentafluoroethylthio, 2-fluoropropylthio, 3-fluoropropylthio, 2-chloropropylthio, 3-chloropropylthio, 2-bromopropylthio, 3-bromopropylthio, 2,2-difluoropropylthio, 2,3-difluoropropylthio, 2,3-dichloropropylthio, 3,3,3-trifluoropropylthio, 3,3,3-trichloropropylthio, 2,2,3,3,3-pentafluoropropylthio, heptafluoropropylthio, 1-(fluoromethyl)-2-fluoroethylthio, 1-(chloromethyl)-2-chloroethylthio, 1-(bromomethyl)-2-bromoethylthio, 4-fluorobutylthio, 4-chlorobutylthio, 4-bromobutylthio, nonafluorobutylthio, 5-fluoropentylthio, 5-chloropentylthio, 5-bromopentylthio, 5-iodopentylthio, undecafluoropentylthio, 6-fluorohexylthio, 6-chlorohexylthio, 6-bromohexylthio, 6-iodohexylthio and dodecafluorohexylthio;

C₁-C₆-alkylsulfinyl (C₁-C₆-alkyl-S(═O)—): for example methylsulfinyl, ethylsulfinyl, propylsulfinyl, 1-methylethylsulfinyl, butylsulfinyl, 1-methylpropylsulfinyl, 2-methylpropylsulfinyl, 1,1-dimethylethylsulfinyl, pentylsulfinyl, 1-methylbutylsulfinyl, 2-methylbutylsulfinyl, 3-methylbutylsulfinyl, 2,2-dimethylpropylsulfinyl, 1-ethylpropylsulfinyl, 1,1-dimethylpropylsulfinyl, 1,2-dimethylpropylsulfinyl, hexylsulfinyl, 1-methylpentylsulfinyl, 2-methylpentylsulfinyl, 3-methylpentylsulfinyl, 4-methylpentylsulfinyl, 1,1-dimethylbutylsulfinyl, 1,2-dimethylbutylsulfinyl, 1,3-dimethylbutylsulfinyl, 2,2-dimethylbutylsulfinyl, 2,3-dimethylbutylsulfinyl, 3,3-dimethylbutylsulfinyl, 1-ethylbutylsulfinyl, 2-ethylbutylsulfinyl, 1,1,2-trimethylpropylsulfinyl, 1,2,2-trimethylpropylsulfinyl, 1-ethyl-1-methylpropylsulfinyl and 1-ethyl-2-methylpropylsulfinyl;

C₁-C₆-haloalkylsulfinyl: a C₁-C₆-alkylsulfinyl radical as mentioned above which is partially or fully substituted by fluorine, chlorine, bromine and/or iodine, i.e., for example, fluoromethylsulfinyl, difluoromethylsulfinyl, trifluoromethylsulfinyl, chlorodifluoromethylsulfinyl, bromodifluoromethylsulfinyl, 2-fluoroethylsulfinyl, 2-chloroethylsulfinyl, 2-bromoethylsulfinyl, 2-iodoethylsulfinyl, 2,2-difluoroethylsulfinyl, 2,2,2-trifluoroethylsulfinyl, 2,2,2-trichloroethylsulfinyl, 2-chloro-2-fluoroethylsulfinyl, 2-chloro-2,2-difluoroethylsulfinyl, 2,2-dichloro-2-fluoroethylsulfinyl, pentafluoroethylsulfinyl, 2-fluoropropylsulfinyl, 3-fluoropropylsulfinyl, 2-chloropropylsulfinyl, 3-chloropropylsulfinyl, 2-bromopropylsulfinyl, 3-bromopropylsulfinyl, 2,2-difluoropropylsulfinyl, 2,3-difluoropropylsulfinyl, 2,3-dichloropropylsulfinyl, 3,3,3-trifluoropropylsulfinyl, 3,3,3-trichloropropylsulfinyl, 2,2,3,3,3-pentafluoropropylsulfinyl, heptafluoropropylsulfinyl, 1-(fluoromethyl)-2-fluoroethylsulfinyl, 1-(chloromethyl)-2-chloroethylsulfinyl, 1-(bromomethyl)-2-bromoethylsulfinyl, 4-fluorobutylsulfinyl, 4-chlorobutylsulfinyl, 4-bromobutylsulfinyl, nonafluorobutylsulfinyl, 5-fluoropentylsulfinyl, 5-chloropentylsulfinyl, 5-bromopentylsulfinyl, 5-iodopentylsulfinyl, undecafluoropentylsulfinyl, 6-fluorohexylsulfinyl, 6-chlorohexylsulfinyl, 6-bromohexylsulfinyl, 6-iodohexylsulfinyl and dodecafluorohexylsulfinyl;

C₁-C₆-alkylsulfonyl (C₁-C₆-alkyl-S(═O)₂—): for example methylsulfonyl, ethylsulfonyl, propylsulfonyl, 1-methylethylsulfonyl, butylsulfonyl, 1-methylpropylsulfonyl, 2-methylpropylsulfonyl, 1,1-dimethylethylsulfonyl, pentylsulfonyl, 1-methylbutylsulfonyl, 2-methylbutylsulfonyl, 3-methylbutylsulfonyl, 1,1-dimethylpropylsulfonyl, 1,2-dimethylpropylsulfonyl, 2,2-dimethylpropylsulfonyl, 1-ethylpropylsulfonyl, hexylsulfonyl, 1-methylpentylsulfonyl, 2-methylpentylsulfonyl, 3-methylpentylsulfonyl, 4-methylpentylsulfonyl, 1,1-dimethylbutylsulfonyl, 1,2-dimethylbutylsulfonyl, 1,3-dimethylbutylsulfonyl, 2,2-dimethylbutylsulfonyl, 2,3-dimethylbutylsulfonyl, 3,3-dimethylbutylsulfonyl, 1-ethylbutylsulfonyl, 2-ethylbutylsulfonyl, 1,1,2-trimethylpropylsulfonyl, 1,2,2-trimethylpropylsulfonyl, 1-ethyl-1-methylpropylsulfonyl and 1-ethyl-2-methylpropylsulfonyl;

C₁-C₆-haloalkylsulfonyl: a C₁-C₆-alkylsulfonyl radical as mentioned above which is partially or fully substituted by fluorine, chlorine, bromine and/or iodine, i.e., for example, fluoromethylsulfonyl, difluoromethylsulfonyl, trifluoromethylsulfonyl, chlorodifluoromethylsulfonyl, bromodifluoromethylsulfonyl, 2-fluoroethylsulfonyl, 2-chloroethylsulfonyl, 2-bromoethylsulfonyl, 2-iodoethylsulfonyl, 2,2-difluoroethylsulfonyl, 2,2,2-trifluoroethylsulfonyl, 2-chloro-2-fluoroethylsulfonyl, 2-chloro-2,2-difluoroethylsulfonyl, 2,2-dichloro-2-fluoroethylsulfonyl, 2,2,2-trichloroethylsulfonyl, pentafluoroethylsulfonyl, 2-fluoropropylsulfonyl, 3-fluoropropylsulfonyl, 2-chloropropylsulfonyl, 3-chloropropylsulfonyl, 2-bromopropylsulfonyl, 3-bromopropylsulfonyl, 2,2-difluoropropylsulfonyl, 2,3-difluoropropylsulfonyl, 2,3-dichloropropylsulfonyl, 3,3,3-trifluoropropylsulfonyl, 3,3,3-trichloropropylsulfonyl, 2,2,3,3,3-pentafluoropropylsulfonyl, heptafluoropropylsulfonyl, 1-(fluoromethyl)-2-fluoroethylsulfonyl, 1-(chloromethyl)-2-chloroethylsulfonyl, 1-(bromomethyl)-2-bromoethylsulfonyl, 4-fluorobutylsulfonyl, 4-chlorobutylsulfonyl, 4-bromobutylsulfonyl, nonafluorobutylsulfonyl, 5-fluoropentylsulfonyl, 5-chloropentylsulfonyl, 5-bromopentylsulfonyl, 5-iodopentylsulfonyl, 6-fluorohexylsulfonyl, 6-bromohexylsulfonyl, 6-iodohexylsulfonyl and dodecafluorohexylsulfonyl.

In a particular embodiment, the variables of the compounds of the formula I have the following meanings, these meanings, both on their own and in combination with one another, being particular embodiments of the compounds of the formula I:

Preference is given to the pyrazolyl-substituted thienyloxypyridines of the formula I in which

R¹, R³ are hydrogen, halogen, cyano, nitro, C₁-C₆-alkyl, C₁-C₆-haloalkyl;

particularly preferably hydrogen, halogen, such as fluorine, chlorine or bromine, or C₁-C₆-alkyl, such as methyl or ethyl;

with particular preference hydrogen, fluorine, chlorine or methyl.

Moreover, preference is given to the pyrazolyl-substituted thienyloxypyridines of the formula I in which

R¹ is hydrogen; and

R³ is hydrogen, halogen, cyano, nitro, C₁-C₆-alkyl or C₁-C₆-haloalkyl;

particularly preferably hydrogen, halogen, such as fluorine, chlorine or bromine, or C₁-C₆-alkyl, such as methyl or ethyl;

with particular preference hydrogen, fluorine, chlorine or methyl.

Moreover, preference is given to the pyrazolyl-substituted thienyloxypyridines of the formula I in which

R¹ is hydrogen, halogen, cyano, nitro, C₁-C₆-alkyl or C₁-C₆-haloalkyl;

particularly preferably hydrogen, halogen, such as fluorine, chlorine or bromine, or C₁-C₆-alkyl, such as methyl or ethyl;

with particular preference hydrogen, fluorine, chlorine or methyl; and

R³ is hydrogen.

Preference is also given to the pyrazolyl-substituted thienyloxypyridines of the formula I in which

R² is hydrogen, halogen, cyano, C₁-C₆-haloalkyl, C₁-C₆-alkoxy, C₁-C₆-alkylamino, di(C₁-C₄-alkyl)amino, C₁-C₆-alkylthio or COR⁷;

particularly preferably hydrogen, halogen, such as, for example, fluorine, chlorine or bromine, cyano or C₁-C₆-haloalkyl, such as, for example, fluoromethyl, chloromethyl, bromomethyl or trifluoromethyl, C₁-C₆-alkoxy, such as, for example, methoxy, or C₁-C₆-alkylthio, such as, for example, methylthio;

particularly preferably hydrogen, fluorine, chlorine, cyano, methoxy or trifluoromethyl.

Preference is furthermore given to the pyrazolyl-substituted thienyloxypyridines of the formula I in which

R² is hydrogen, halogen, cyano, C₁-C₆-alkoxy, C₁-C₆-alkenyloxy, C₁-C₆-alkynyloxy, C₁-C₆-haloalkoxy, C₁-C₆-alkylamino, di(C₁-C₄-alkyl)amino, C₁-C₆-alkylthio, C₁-C₆-haloalkylthio, C₁-C₆-alkylsulfinyl, C₁-C₆-haloalkylsulfinyl, C₁-C₆-alkylsulfonyl or C₁-C₆-haloalkylsulfonyl;

very preferably hydrogen, halogen, cyano, C₁-C₆-alkoxy, C₁-C₆-alkylamino, di(C₁-C₄-alkyl)amino or C₁-C₆-alkylthio;

with particular preference hydrogen, halogen, such as, for example, fluorine, chlorine or bromine, cyano, C₁-C₆-alkoxy, such as, for example, methoxy, or C₁-C₆-alkylthio, such as, for example, methylthio;

with particular preference hydrogen, fluorine, chlorine, cyano or methoxy.

Preference is also given to the pyrazolyl-substituted thienyloxypyridines of the formula I in which

R² is C₂-C₆-alkenyl, C₂-C₆-alkynyl, C₁-C₆-haloalkyl, C₂-C₆-haloalkenyl, C₂-C₆-haloalkynyl, C₁-C₆-alkoxy-C₁-C₄-alkyl or COR⁷;

very preferably C₁-C₆-haloalkyl or COR⁷;

particularly preferably C₁-C₆-haloalkyl, such as, for example, fluoromethyl, chloromethyl, bromomethyl or trifluoromethyl;

with particular preference trifluoromethyl.

Preference is furthermore given to the pyrazolyl-substituted thienyloxypyridines of the formula I in which

R¹, R³ are hydrogen, halogen, cyano, nitro, C₁-C₆-alkyl or C₁-C6-haloalkyl;

particularly preferably hydrogen, halogen, such as fluorine, chlorine or bromine, C₁-C₆-alkyl, such as methyl or ethyl, C₁-C₆-haloalkyl, such as fluoromethyl, chloromethyl or trifluoromethyl;

with particular preference hydrogen, fluorine, chlorine or methyl; and

R² is hydrogen, halogen, cyano, C₁-C₆-haloalkyl, C₁-C₆-alkoxy, C₁-C₆-alkylamino, di(C₁-C₄-alkyl)amino, C₁-C₆-alkylthio or COR⁷;

particularly preferably hydrogen, halogen, such as, for example, fluorine, chlorine or bromine, cyano, C₁-C₆-haloalkyl, such as, for example, fluoromethyl, chloromethyl, bromomethyl or trifluoromethyl, C₁-C₆-alkoxy, such as, for example, methoxy, or C₁-C₆-alkylthio, such as, for example, methylthio;

with particular preference hydrogen, fluorine, chlorine, cyano, methoxy or trifluoromethyl.

Preference is also given to the pyrazolyl-substituted thienyloxypyridines of the formula I in which

R¹ is hydrogen, halogen, cyano, nitro, C₁-C₆-alkyl or C₁-C₆-haloalkyl;

particularly preferably hydrogen, halogen, such as, fluorine, chlorine or bromine, C₁-C₆-alkyl, such as methyl or ethyl, C₁-C₆-haloalkyl such as fluoromethyl, chloromethyl or trifluoromethyl;

with particular preference hydrogen, fluorine, chlorine or methyl; and

R² is hydrogen, halogen, cyano, C₁-C₆-haloalkyl, C₁-C₆-alkoxy, C₁-C₆-alkylamino, di(C₁-C₄-alkyl)amino, C₁-C₆-alkylthio or COR⁷;

particularly preferably hydrogen, halogen, such as, for example, fluorine, chlorine or bromine, cyano, C₁-C₆-haloalkyl, such as, for example, fluoromethyl, chloromethyl, bromomethyl or trifluoromethyl, C₁-C₆-alkoxy, such as, for example, methoxy, or C₁-C₆-alkylthio, such as, for example, methylthio;

with particular preference hydrogen, fluorine, chlorine, cyano, methoxy or trifluoromethyl; and

R³ is hydrogen.

In addition, preference is given to the pyrazolyl-substituted thienyloxypyridines of the formula I in which

R¹ is hydrogen;

R² is hydrogen, halogen, cyano, C₁-C₆-haloalkyl, C₁-C₆-alkoxy, C₁-C₆-alkylamino, di(C₁-C₄-alkyl)amino, C₁-C₆-alkylthio or COR⁷;

particularly preferably hydrogen, halogen, such as, for example, fluorine, chlorine or bromine, cyano, C₁-C₆-haloalkyl, such as, for example, fluoromethyl, chloromethyl, bromomethyl or trifluoromethyl, C₁-C₆-alkoxy, such as, for example, methoxy, or C₁-C₆-alkylthio, such as, for example, methylthio;

with particular preference hydrogen, fluorine, chlorine, cyano, methoxy or trifluoromethyl; and

R³ is hydrogen.

Preference is furthermore given to the pyrazolyl-substituted thienyloxypyridines of the formula I in which

R¹ is halogen, such as, for example, fluorine, chlorine or bromine;

particularly preferably fluorine or chlorine;

R² is hydrogen, halogen, cyano, C₁-C₆-haloalkyl, C₁-C₆-alkoxy, C₁-C₆-alkylamino, di(C₁-C₄-alkyl)amino, C₁-C₆-alkylthio or COR⁷;

particularly preferably hydrogen, halogen, such as, for example, fluorine, chlorine or bromine, cyano, C₁-C₆-haloalkyl, such as, for example, fluoromethyl, chloromethyl, bromomethyl or trifluoromethyl, C₁-C₆-alkoxy, such as, for example, methoxy, or C₁-C₆-alkylthio, such as, for example, methylthio;

with particular preference hydrogen, fluorine, chlorine, cyano, methoxy or trifluoromethyl; and

R³ is halogen, such as, for example, fluorine, chlorine or bromine;

particularly preferably fluorine or chlorine.

Preference is also given to the pyrazolyl-substituted thienyloxypyridines of the formula I in which, in each case independently of one another,

R⁴, R⁵, R⁶ are hydrogen, halogen, cyano, C₁-C₆-alkyl, C₁-C₆-haloalkyl, C₁-C₆-alkoxy, C₁-C₆-haloalkoxy, C₁-C₆-alkylthio, C₁-C₆-alkylsulfonyl, C₁-C₆-haloalkylsulfonyl;

particularly preferably hydrogen, halogen, cyano, C₁-C₆-alkyl, C₁-C₆-haloalkyl, C₁-C₆-haloalkoxy, C₁-C₆-alkylsulfonyl or C₁-C₆-haloalkylsulfonyl;

with particular preference hydrogen, halogen, such as fluorine, chlorine or bromine, C₁-C₆-haloalkyl, such as trifluoromethyl, trichloromethyl or difluoromethyl, C₁-C₆-haloalkoxy, such as difluoromethoxy or trifluoromethoxy;

very preferably hydrogen, fluorine, chlorine, trifluoromethyl or difluoromethoxy.

Preference is also given to the pyrazolyl-substituted thienyloxypyridines of the formula I in which R⁶ is hydrogen and, in each case independently of one another,

R⁴, R⁵ are hydrogen, halogen, C₁-C₆-alkyl or C₁-C₆-haloalkyl;

particularly preferably hydrogen, chlorine, methyl or trifluoromethyl.

Preference is also given to the pyrazolyl-substituted thienyloxypyridines of the formula I in which

R⁷ is hydrogen, C₁-C₆-alkyl or C₁-C₆-alkoxy, such as, for example, methoxy or ethoxy;

particularly preferably hydrogen, methoxy or ethoxy.

Particular preference is also given to the pyrazolyl-substituted thienyloxypyridines of the formula I in which the thienyl radical is attached in the 3-position via the oxygen atom to the pyridine skeleton and substituted by R⁴ and R⁵ in the 4- and 5-positions, respectively.

Particular preference is also given to the pyrazolyl-substituted thienyloxypyridines of the formula I in which the thienyl radical is attached in the 2- and 5-position via the oxygen atom to the pyridine skeleton and substituted by R⁴ and R⁵ in the 4- and 5-positions, respectively.

Preference is also given to the pyrazolyl-substituted thienyloxypyridines of the formula I in which R⁵ and R⁶ are hydrogen and

R⁴ is halogen, cyano, C₁-C₆-alkyl, C₁-C₆-haloalkyl or C₁-C₆-haloalkoxy;

particularly preferably halogen or C₁-C₆-haloalkyl;

very preferably fluorine, chlorine or trifluoromethyl.

Particular preference is also given to the pyrazolyl-substituted thienyloxypyridines of the formula I in which the thienyl radical is attached in the 3-position via the oxygen atom to the pyridine skeleton and substituted by R⁴ in the 5-position.

Particular preference is also given to the pyrazolyl-substituted thienyloxypyridines of the formula I in which the thienyl radical is attached in the 2-position via the oxygen atom to the pyridine skeleton and substituted by R⁴ in the 5-position.

Most preference is given to compounds of the formula Ia (where R⁴=5—CF₃, R⁵=H, R⁶=H; the thienyl radical is attached in the 3-position via an oxygen atom to the pyridine skeleton), in particular to the compounds Ia.1 to Ia.52 of Table 1, where the definitions of the variables R¹ to R⁶ play a particular role for the compounds according to the invention, not only in combination with one another but in each case also on their own.

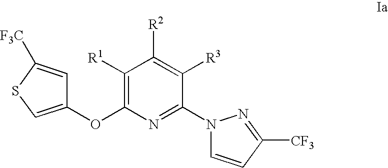

TABLE 1

| No. | R¹ | R² | R³ |
|---|---|---|---|
| Ia.1 | H | H | H |
| Ia.2 | H | Cl | H |
| Ia.3 | H | CN | H |
| Ia.4 | H | CH₂Br | H |
| Ia.5 | H | CF₃ | H |
| Ia.6 | H | OCH₃ | H |
| Ia.7 | H | OCH₃ | CN |
| Ia.8 | CN | OCH₃ | H |
| Ia.9 | H | SCH₃ | H |
| Ia.10 | H | CHO | H |
| Ia.11 | H | CO₂CH₃ | H |
| Ia.12 | H | H | Cl |
| Ia.13 | H | H | CN |
| Ia.14 | H | H | NO₂ |
| Ia.15 | H | H | CH₃ |
| Ia.16 | H | H | CH₂Cl |
| Ia.17 | H | H | CF₃ |
| Ia.18 | Cl | H | H |
| Ia.19 | CN | H | H |
| Ia.20 | NO₂ | H | H |
| Ia.21 | CH₃ | H | H |
| Ia.22 | CH₂Cl | H | H |
| Ia.23 | CF₃ | H | H |
| Ia.24 | F | H | F |
| Ia.25 | F | F | F |
| Ia.26 | F | Cl | F |
| Ia.27 | F | Br | F |
| Ia.28 | F | CN | F |
| Ia.29 | F | CF₃ | F |
| Ia.30 | F | OCH₃ | F |
| Ia.31 | F | SCH₃ | F |
| Ia.32 | F | N(CH₃)₂ | F |
| Ia.33 | F | CO₂H | F |
| Ia.34 | F | CO₂CH₃ | F |
| Ia.35 | Cl | H | Cl |
| Ia.36 | Cl | F | Cl |
| Ia.37 | Cl | Cl | Cl |
| Ia.38 | Cl | CN | Cl |
| Ia.39 | Cl | CF₃ | Cl |
| Ia.40 | Cl | OCH₃ | Cl |
| Ia.41 | Cl | SCH₃ | Cl |
| Ia.42 | NO₂ | H | NO₂ |
| Ia.43 | CH₃ | H | CH₃ |
| Ia.44 | CF₃ | H | CF₃ |
| Ia.45 | Cl | H | CF₃ |
| Ia.46 | NO₂ | H | CF₃ |
| Ia.47 | CH₃ | H | CF₃ |
| Ia.48 | CF₃ | H | Cl |
| Ia.49 | CF₃ | H | NO₂ |
| Ia.50 | CF₃ | H | CH₃ |
| Ia.51 | F | H | CN |
| Ia.52 | CN | H | F |

Most preference is also given to the compounds of the formula Ib, in particular to the compounds Ib.1 to Ib.52 which differ from the corresponding compounds Ia.1 to Ia.52 in that R⁴ in the 5-position is chlorine.

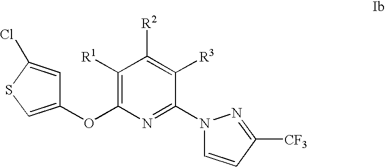

Most preference is also given to the compounds of the formula Ic, in particular to the compounds Ic.1 to Ic.52 which differ from the corresponding compounds Ia.1 to Ia.52 in that the thienyl radical is attached in the 2-position via the oxygen atom to the pyridine skeleton.

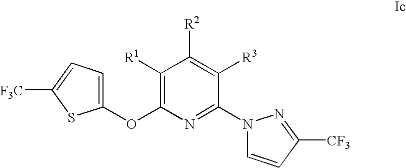

Most preference is also given to the compounds of the formula Id, in particular to the compounds Id.1 to Id.52 which differ from the corresponding compounds Ia.1 to Ia.52 in that R⁴ in the 5-position is chlorine and the thienyl radical is attached in the 2-position via the oxygen atom to the pyridine skeleton.

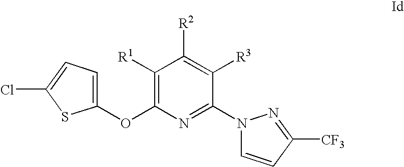

Most preference is also given to the compounds of the formula Ie, in particular to the compounds Ie.1 to Ie.52 which differ from the corresponding compounds Ia.1 to Ia.52 in that R⁴ in the 4-position is trifluoromethyl and the thienyl radical is attached in the 2-position via the oxygen atom to the pyridine skeleton.

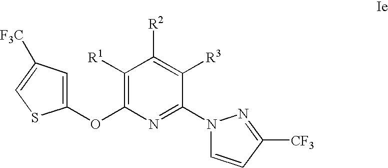

Most preference is also given to the compounds of the formula If, in particular to the compounds If.1 to If.52 which differ from the corresponding compounds Ia.1 to Ia.52 in that R⁴ in the 4-position is chlorine and the thienyl radical is attached in the 2-position via the oxygen atom to the pyridine skeleton.

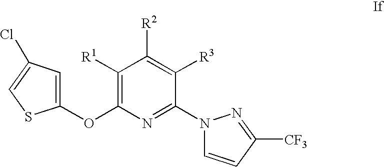

The pyrazolyl-substituted thienyloxypyridines of the formula I can be obtained by various methods, for example by the processes below.

Process A

The 3-trifluoromethyl-1H-pyrazol-1-yl-substituted pyridines of the formula III are obtained from pyridines of the formula V by reaction with 3-trifluoromethyl-1H-pyrazole IV. L¹ and L² are nucleophilically displaceable leaving groups, such as halogen, for example fluorine, chlorine and bromine, C₁-C₄-alkylsulfonyl, such as, for example, methylsulfonyl, C₁-C₄-alkylsulfonyloxy, such as, for example, methylsulfonyloxy, C₁-C₄-haloalkylsulfonyloxy or trialkylammonium, preferably fluorine, chlorine or bromine, C₁-C₄-alkylsulfonyl, such as, for example, methylsulfonyl, or C₁-C₄-haloalkylsulfonyloxy, such as, for example, trifluoromethylsulfonyloxy. These compounds are then reacted with hydroxythiophenes of the formula II to give pyrazole-substituted thienyloxypyridines of the formula I:

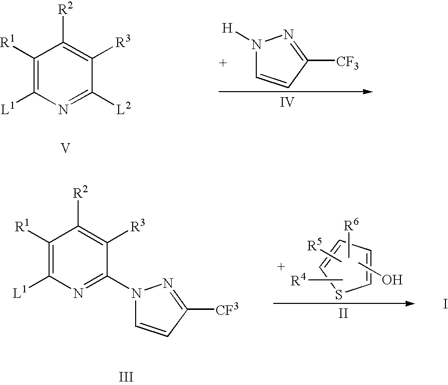

The conversion of pyridines of the formula V into 3-trifluoromethyl-1H-pyrazol-1-yl-substituted pyridines of the formula III is usually carried out at 0° C.-200° C., preferably at 10° C.-100° C., in an inert organic solvent in the presence of a base [cf. WO 98/40379; EP 1 101 764].

Suitable solvents are aliphatic hydrocarbons, such as pentane, hexane, cyclohexane and mixtures of C₅-C₈-alkanes, ethers, such as diethyl ether, diisopropyl ether, tert-butyl methyl ether, dioxane, anisole and tetrahydrofuran, nitriles, such as acetonitrile and propionitrile, and also dimethyl sulfoxide, dimethylformamide and dimethylacetamide, particularly preferably acetonitrile and dimethylformamide.

Also suitable for use as solvents are aromatic hydrocarbons, such as, for example, toluene and xylene.

It is also possible to use mixtures of the solvents mentioned.

Suitable bases are, in general, inorganic compounds, such as alkali metal and alkaline earth metal hydroxides, such as lithium hydroxide, sodium hydroxide, potassium hydroxide and calcium hydroxide, alkali metal and alkaline earth metal hydrides, such as lithium hydride, sodium hydride, potassium hydride and calcium hydride, alkali metal amides, such as lithium amide, sodium amide and potassium amide, alkali metal and alkaline earth metal carbonates, such as lithium carbonate, potassium carbonate and calcium carbonate, and also alkali metal and alkaline earth metal alkoxides, such as sodium methoxide, sodium ethoxide, potassium ethoxide, potassium tert-butoxide and potassium tert-pentoxide; organic bases, for example tertiary amines, such as trimethylamine, triethylamine, diisopropylethylamine and N-methylpiperidine, pyridine, substituted pyridines, such as collidine, lutidine and 4-dimethylaminopyridine, and also bicyclic amines. Particular preference is given to potassium carbonate, sodium hydride, potassium tert-butoxide and potassium tert-pentoxide.

Cesium carbonate is also preferred as a base.

The bases are generally employed in equimolar amounts; however, it is also possible to employ them in excess or, if appropriate, as solvent.

The starting materials are generally reacted with one another in equimolar amounts. In terms of yield, it may be advantageous to employ an excess of V, based on IV.

It may be advantageous to employ catalytic amounts of copper or Cu(I) salts, such as, for example, CuBr or Cu triflate.

The conversion of 3-trifluoromethyl-1H-pyrazol-1-yl-substituted pyridines of the formula III into pyrazolyl-substituted thienyloxypyridines of the formula I is usually carried out at 50° C.-200° C., preferably at 50° C.-150° C., in an inert organic solvent in the presence of a base [cf. WO 98/40379; EP 1 101 764].

Suitable solvents are aliphatic hydrocarbons, such as pentane, hexane, cyclohexane and mixtures of C₅-C₈-alkanes, ethers, such as diethyl ether, diisopropyl ether, tert-butyl methyl ether, dioxane, anisole, tetrahydrofuran and diethylene glycol dimethyl ether, nitriles, such as acetonitrile and propionitrile, and also dimethyl sulfoxide, dimethylformamide, dimethylacetamide, N-methylpyrrolidone and sulfolane, particularly preferably acetonitrile, diethylene glycol dimethyl ether, dimethylformamide, N-methylpyrrolidone and sulfolane.

It is also possible to use mixtures of the solvents mentioned.

Suitable bases are, in general, inorganic compounds, such as alkali metal and alkaline earth metal hydroxides, such as lithium hydroxide, sodium hydroxide, potassium hydroxide and calcium hydroxide, alkali metal and alkaline earth metal hydrides, such as lithium hydride, sodium hydride, potassium hydride and calcium hydride, alkali metal amides, such as lithium amide, sodium amide and potassium amide, alkali metal and alkaline earth metal carbonates, such as lithium carbonate, potassium carbonate and calcium carbonate, and also alkali metal and alkaline earth metal alkoxides, such as sodium methoxide, sodium ethoxide, potassium ethoxide, potassium tert-butoxide and potassium tert-pentoxide; organic bases, for example tertiary amines, such as trimethylamine, triethylamine, diisopropylethylamine and N-methylpiperidine, pyridine, substituted pyridines, such as collidine, lutidine and 4-dimethylaminopyridine, and also bicyclic amines. Particular preference is given to potassium carbonate, sodium hydride, potassium tert-butoxide and potassium tert-pentoxide.

The bases are generally employed in equimolar amounts; however, they can also be used in excess or, if appropriate, as solvent.

The starting materials are generally reacted with one another in equimolar amounts. In terms of yield, it may be advantageous to employ an excess of II, based on III.

The starting materials required for preparing the compounds I are known from the literature or can be prepared in accordance with the literature cited [cf. EP 1 101 764].

The reaction mixtures are worked up in a customary manner, for example by mixing with water, separating the phases and, if appropriate, purifying the crude products by chromatography. Some of the intermediates and end products are obtained in the form of colorless or slightly brownish, viscous oils which, under reduced pressure and at moderately elevated temperature, can be freed from volatile fractions or purified. If the intermediates and end products are obtained as solids, purification can also be effected by recrystallization or digestion.

Process B

A dihalopyridine of the formula V (where L¹=Hal and L²=Hal′) is reacted with sodium mercaptan or potassium mercaptan of the formula VIII to give pyridines of the formula VII. Here, R^{a} is C₁-C₆-alkyl, preferably methyl. The pyridines of the formula VII can then be reacted with a pyrazole of the formula IV to give pyrazolyl-substituted pyridines of the formula VI:

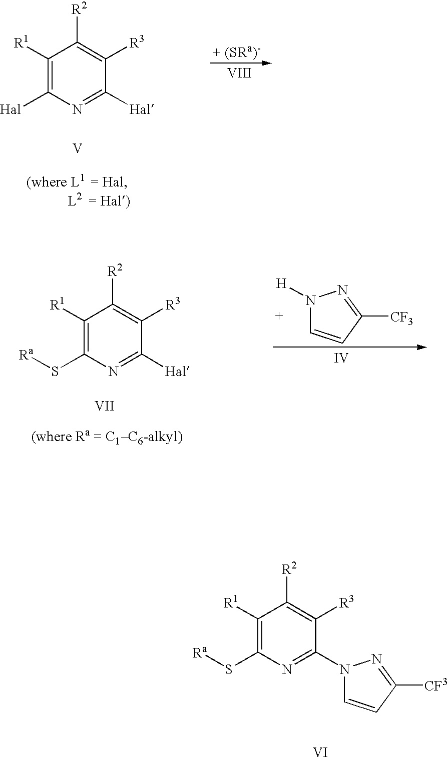

The conversion into pyridines of the formula VII is usually carried out at 0° C.-80° C. in an inert organic solvent [cf. WO 98/40379].

Suitable solvents are ethers, such as diethyl ether, diisopropyl ether, tert-butyl methyl ether, dioxane, anisole and tetrahydrofuran, particularly preferably tetrahydrofuran.

It is also possible to use mixtures of the solvents mentioned.

The starting materials are generally reacted with one another in equimolar amounts.

Work-up can be carried out in a manner known per se to afford the product.

The conversion of pyridines of the formula VII into pyrazole-substituted pyridines of the formula VI is usually carried out at 50° C.-200° C., preferably at 50° C.-150° C., analogously to the conversion of V into III (cf. process A).

The pyrazolyl-substituted pyridines of the formula VI are then oxidized to give compounds of the formula III (where L¹=SO₂R^{a}). By further reaction with hydroxythiophenes of the formula II, the pyrazolyl-substituted thienyloxypyridines of the formula I are obtained:

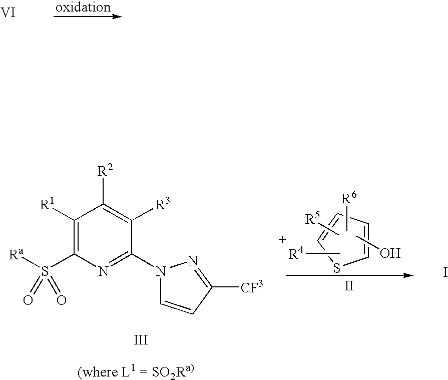

The oxidation is usually carried out at 0° C.-100° C., preferably at 25° C., in an inert organic solvent [cf. J. March, Organic Chemistry, 1992, 1201-1203].

Suitable oxidizing agents are, for example, metachloroperbenzoic acid, peroxyacetic acid, trifluoroperoxyacetic acid, hydrogen peroxide, sodium periodate or Oxone®. It may be advantageous to carry out the reaction in the presence of a catalyst, for example sodium tungstate.

Suitable solvents are halogenated hydrocarbons, such as methylene chloride, chloroform and chlorobenzene, alcohols, such as methanol, ethanol, n-propanol, isopropanol, n-butanol and tert-butanol.

The starting materials are generally reacted with one another in equimolar amounts. In terms of yield, it may be advantageous to employ an excess of oxidizing agent, based on VI.

Work-up can be carried out in a manner known per se to afford the product.

The reaction of compounds of the formula III with hydroxythiophenes of the formula II is carried out under the same conditions as the conversion of III into I (cf. process A).

Process C

It is also possible to synthesize the nitrogen heterocycle directly from a corresponding aminopyridine. This gives pyrazolyl-substituted pyridines which can then be modified further by the reactions shown above. By way of example, this may be demonstrated using the conversion of the aminopyridines of the formula IX into the 3-trifluoromethyl-1H-pyrazol-1-yl-substituted pyridines of the formula III (where L¹=chlorine). However, the heterocycle can also be constructed at a different stage of the variants A, B and D to F shown.

The aminopyridine of the formula IX is initially converted into the diazonium compound, giving, after hydrogenation, the corresponding pyridinehydrazine derivative. This is then reacted with 1,3-dicarbonyl compounds, enol esters or 1-alkynyl ketones in a cyclocondensation to give the desired pyrazole:

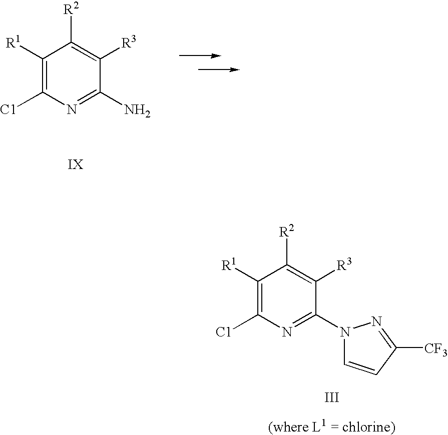

The resulting 3-trifluoromethyl-1H-pyrazol-1-yl-substituted pyridines of the formula III can then be modified further by the reactions presented here.

The abovementioned reactions are generally known from the literature and described, inter alia, in T. Eicher, S. Hauptmann, Chemie der Heterocyclen [Chemistry of heterocycles], 1994, 183; A. S. Tomcufcik, L. N. Starker, The Chemistry of Heterocyclic Compounds, Pyridine and its Derivatives part 3, 1962, 34-35.

Process D

In this variant, pyridines of the formula XII are initially reacted with a pyrazole of the formula IV under the same reaction conditions which can also be used to convert V into III (cf. process A). The product is then oxidized giving a pyridine N-oxide of the formula X and, after halogenation, a 3-trifluoromethyl-1H-pyrazol-1-yl-substituted pyridine of the formula III where L¹=Hal is obtained. Pyrazolyl-substituted thienyloxypyridines of the formula I are obtained by analogous reaction of the 3-trifluoromethyl-1H-pyrazol-1-yl-substituted pyridines of the formula III with hydroxythiophenes of the formula II, as described in process A.

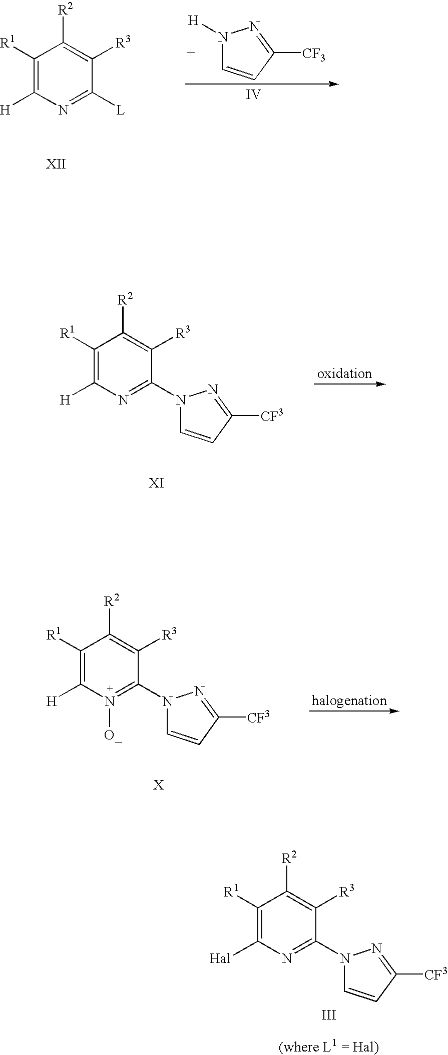

The oxidation of the pyridines of the formula XI to give pyridine N-oxides of the formula X is usually carried out at 0° C.-100° C., preferably at 0° C.-25° C., in an inert organic solvent [cf. G. C. Finger et al., J. Am. Chem. Soc. 81 (1959), 2674-2675; M. Tiecco et al., Tetrahedron 42 (1986), 1475-1485].

Suitable oxidizing agents are, for example, metachloroperbenzoic acid, peroxyacetic acid or hydrogen peroxide.

It may be advantageous to carry out the reaction in the presence of a catalyst, for example sodium tungstate.

Suitable solvents are halogenated hydrocarbons, such as methylene chloride, chloroform and chlorobenzene, and alcohols, such as methanol, ethanol, n-propanol, isopropanol, n-butanol and tert-butanol.

Trifluoroacetic acid is also a suitable solvent.

The starting materials are generally reacted with one another in equimolar amounts. In terms of yield, it may be advantageous to employ an excess of oxidizing agent, based on XI.

Work-up can be carried out in a manner known per se to afford the product.

The halogenation of the pyridine N-oxides of the formula X to give 3-trifluoromethyl-1H-pyrazol-1-yl-substituted pyridines of the formula III where L¹=Hal is usually carried out at 25° C.-200° C., preferably at 80° C.-150° C., in an inert organic solvent [cf. H. E. Mertel, The Chemistry of Heterocyclic Compounds, Pyridine and its Derivatives part 2, 1961, 305-307].

Suitable halogenating agents are, for example, phosphorus oxytrichloride, phosphorus oxytribromide or sulfuryl chloride.

Thionyl chloride is also a suitable halogenating agent.

Suitable solvents are aromatic hydrocarbons, such as toluene and o-, m- and p-xylene.

The starting materials are generally reacted with one another in equimolar amounts. In terms of yield, it may be advantageous to employ an excess of halogenating agent, based on X.

Work-up can be carried out in a manner known per se to afford the product.

Process E

Thienyloxypyridines of the formula XIII are obtained by reacting pyridines of the formula V with hydroxythiophenes of the formula II (cf. EP 955 300). This reaction is usually carried out at 25° C.-200° C., preferably at 80° C.-150° C., analogously to the reaction conditions described for the conversion of III into I (cf. process A). The thienyloxypyridines of the formula XIII are then reacted, analogously to the conversion of V into III (cf. process A), with pyrazole derivatives of the formula IV (cf. EP 1 101 764):

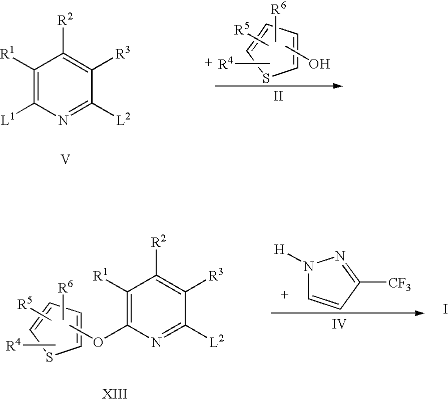

Alternatively, the conversion of XIII into I can also be carried out catalytically using nickel or palladium. In this case, the reaction is usually carried out at 25° C.-130° C. in an inert organic solvent in the presence of a base [cf. B. Gradel et al., Tetrahedron Lett. 42 (2001), 5689-5692; J. F. Hartwig et al., J. Am. Chem. Soc. 120 (1998), 827-828].

Here, L² is usually a halogen atom, such as, for example, chlorine, bromine or iodine, or another leaving group, such as, for example, trifluoromethylsulfonyloxy.

Suitable catalysts are, for example, nickel or palladium ligand complexes in which the metal is present in oxidation stage 0, preferably nickel(II) or palladium(II) salts. The reaction with nickel(II) or palladium(II) salts is preferably carried out in the presence of complex ligands.

Suitable nickel(0) complexes are, for example, nickel carbene complexes.

Suitable palladium(0) complex ligands are, for example, tetrakis(triphenylphosphine)palladium, palladium(diphenylphosphineferrocene) dichloride {[PdCl₂(dppf)]} or tris-(dibenzylideneacetone)dipalladium (Pd₂dba)₃.

Suitable nickel(II) salts are, for example, nickel acetate and nickel acetylacetonate.

Suitable palladium(II) salts are, for example, palladium acetate and palladium chloride. The reaction is preferably carried out in the presence of complex ligands, such as, for example, diphenylphosphineferrocene (dppf).

The complex nickel salts can be prepared in a manner known per se from commercially available nickel salts, such as nickel chloride or nickel acetate, and the corresponding phosphines, such as, for example, triphenylphosphine or 1,2-bis(triphenylphosphino)ethane, or commercially available imidazolinium salts. Many complex nickel salts are also commercially available.

The complex palladium salts can be prepared in a manner known per se from commercially available palladium salts, such as palladium chloride or palladium acetate, and the corresponding phosphines, such as, for example, triphenylphosphine or 1,2-bis(diphenylphosphino)ethane. Many complex palladium salts are also commercially available. Preferred palladium salts are [(R)-(+)-2,2′-bis(diphenylphosphino)-1,1′-binaphthyl]palladium(II) chloride, bis(triphenylphosphine)palladium(II) acetate and, in particular, bis(triphenylphosphine)palladium(II) chloride.

The catalyst is generally employed in a concentration of from 0.05 to 5 mol %, preferably from 1 to 3 mol %.

Suitable solvents are aromatic hydrocarbons, such as toluene, o-, m- and p-xylene, ethers, such as diethyl ether, diisopropyl ether, tert-butyl methyl ether, dioxane, anisole and tetrahydrofuran, and also dimethylformamide.

Suitable bases are, in general, inorganic compounds, such as alkali metal and alkaline earth metal hydroxides, such as lithium hydroxide, sodium hydroxide, potassium hydroxide and calcium hydroxide, alkali metal and alkaline earth metal hydrides, such as lithium hydride, sodium hydride, potassium hydride and calcium hydride, alkali metal and alkaline earth metal carbonates, such as sodium carbonate, potassium carbonate and cesium carbonate, and also alkali metal and alkaline earth metal alkoxides, such as sodium methoxide, sodium ethoxide, potassium ethoxide and potassium tert-butoxide.

The bases are generally employed in equimolar amounts.

The starting materials are generally reacted with one another in equimolar amounts. In terms of yield, it may be advantageous to employ an excess of IV, based on XIII.

Work-up can be carried out in a manner known per se to afford the product.

Process F

Alternatively to process E, dithienyloxy-substituted pyridines of the formula XIV are obtained by reacting pyridines of the formula V with an excess of the hydroxythiophene of the formula II (cf. EP-A-955 300). The reaction is preferably carried out using a double-equimolar ratio of II to V. This reaction is carried out analogously to the reaction conditions described for the conversion of III into I (cf. process A). The dithienyloxy-substituted pyridines of the formula XIV are then, usually at 25° C.-200° C., preferably at 80° C.-150° C., reacted analogously to the conversion of V into III (cf. process A) with pyrazoles of the formula IV (cf. EP 1 101 764):

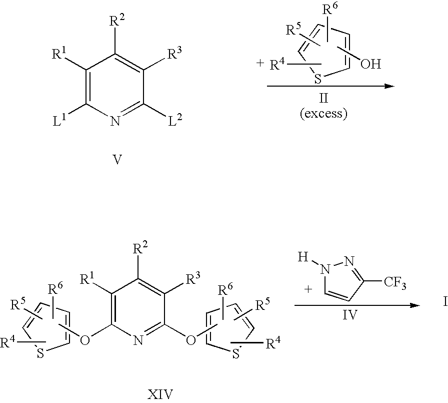

3-Trifluoromethyl-1H-pyrazol-1-yl-substituted pyridine derivatives of the formula III

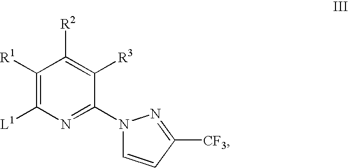

where R¹, R² and R³ are as defined for compounds of the formula I and L¹ is a nucleophilically displaceable leaving group, such as halogen, for example chlorine, bromine or iodine, C₁-C₄-alkylsulfonyl, C₁-C₄-alkylsulfonyloxy, C₁-C₄-haloalkylsulfonyloxy or trialkylammonium, preferably fluorine, chlorine or bromine, C₁-C₄-alkylsulfonyl, such as, for example, methylsulfonyl, or C₁-C₄-haloalkylsulfonyloxy, such as, for example, trifluoromethylsulfonyloxy, also form part of the subjectmatter of the present invention.

The particularly preferred embodiments of the intermediates with respect to the variables correspond to those of the radicals R¹, R² and R³ of the formula I.

Particular preference is given to compounds of the formula III in which

R¹, R³ are hydrogen, halogen, cyano, C₁-C₆-alkyl or C₁-C₆-haloalkyl;

particularly preferably hydrogen, halogen, such as fluorine, chlorine or bromine, C₁-C₆-alkyl, such as methyl or ethyl;

with particular preference hydrogen, fluorine, chlorine or methyl; and

R² is hydrogen, halogen, cyano, C₁-C₆-haloalkyl, C₁-C₆-alkoxy, C₁-C₆-alkylthio or COR⁷;

particularly preferably hydrogen, halogen, such as, for example, fluorine, chlorine or bromine, cyano, C₁-C₆-haloalkyl, such as, for example, fluoromethyl, chloromethyl, bromomethyl or trifluoromethyl, C₁-C₆-alkoxy, such as, for example, methoxy, or C₁-C₆-alkylthio;

with particular preference hydrogen, fluorine, chlorine, cyano, methoxy or trifluoromethyl.

Thienyloxypyridine derivatives of the formula XIII

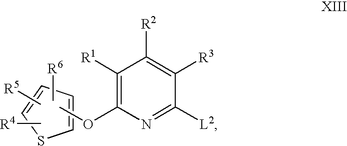

where R¹, R², R³, R⁴, R⁵ and R⁶ are as defined for compounds of the formula I and L² is a nucleophilically displaceable leaving group, such as halogen, for example fluorine, chlorine or bromine, C₁-C₄-alkylsulfonyl, C₁-C₄-alkylsulfonyloxy, such as, for example, methylsulfonyloxy, C₁-C₄-haloalkylsulfonyloxy or trialkylammonium, preferably fluorine, chlorine or bromine, C₁-C₄-alkylsulfonyl, such as, for example, methylsulfonyl, or C₁-C₄-haloalkylsulfonyloxy, such as, for example, trifluoromethylsulfonyloxy, also form part of the subject matter of the present invention.

The particularly preferred embodiments of the compounds of the formula XIII with respect to the variables correspond to those of the radicals R¹, R², R³, R⁴, R⁵ and R⁶ of the formula I.

Particular preference is given to the compounds of the formula XIII, in which L² is halogen, such as, for example, fluorine or chlorine.

Preference is given to compounds of the formula XIII, in which

R¹, R³ are hydrogen, halogen, cyano, C₁-C₆-alkyl or C₁-C₆-haloalkyl;

particularly preferably hydrogen, halogen, such as fluorine, chlorine or bromine, C₁-C₆-alkyl, such as methyl or ethyl;

with particular preference hydrogen, fluorine, chlorine or methyl;

R² is hydrogen, halogen, cyano, C₁-C₆-haloalkyl, C₁-C₆-alkoxy, C₁-C₆-alkylthio or COR⁷;

particularly preferably hydrogen, halogen, such as for example fluorine, chlorine or bromine, cyano or C₁-C₆-haloalkyl, such as, for example, fluoromethyl, chloromethyl, bromomethyl or trifluoromethyl, C₁-C₆-alkoxy, such as, for example, methoxy, or C₁-C₆-alkylthio;

with particular preference hydrogen, fluorine, chlorine, cyano, methoxy or trifluoromethyl; and

R⁴, R⁵, R⁶ are hydrogen, halogen, cyano, C₁-C₆-alkyl, C₁-C₆-haloalkyl, C₁-C₆-alkoxy, C₁-C₆-haloalkoxy, C₁-C₆-alkylthio, C₁-C₆-alkylsulfonyl or C₁-C₆-haloalkylsulfonyl;

particularly preferably hydrogen, halogen, cyano, C₁-C₆-alkyl, C₁-C₆-haloalkyl, C₁-C₆-haloalkoxy, C₁-C₆-alkylsulfonyl or C₁-C₆-haloalkylsulfonyl;

with particular preference hydrogen, halogen, C₁-C₆-haloalkyl or C₁-C₆-haloalkoxy;

very preferably hydrogen, fluorine, chlorine, trifluoromethyl or difluoromethoxy.

### PREPARATION EXAMPLES

In accordance with process E:

2,3,5-Trifluoro-6-(5-trifluoromethyl-3-thienyloxy)pyridine

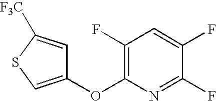

3 g (19.9 mmol) of 2,3,5,6-tetrafluoropyridine, 3.34 g (19.9 mmol) of 5-trifluoromethyl-3-hydroxythiophene and 5.48 g (39.7 mmol) of potassium carbonate in 30 ml of DMF were stirred at room temperature for 12 h. The mixture was diluted with 200 ml of water and then extracted with diethyl ether. The organic phase was washed and dried and the solvent was removed. This gave 4.86 g (16.3 mmol, 82%) of the title compound.

1H-NMR (400 MHz, CDCl₃): δ=7.3 (s, 1H), 7.35 (s, 1H), 7.5 (m, 1H).

3,5-Difluoro-2-(3-trifluoromethyl-1H-pyrazol-1-yl)-6-(5-trifluoromethyl-3-thienyloxy)pyridine

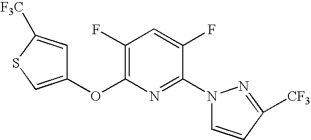

A mixture of 0.2 g (0.67 mmol) of 2,3,5-trifluoro-6-(5-trifluoromethyl-3-thienyloxy)pyridine, 0.08 g (0.59 mmol) of 3-trifluoromethyl-1H-pyrazole and 0.14 g (0.1 mmol) of potassium carbonate in 20 ml of N,N-dimethylformamide (DMF) was heated at 80° C. for 12 h. The mixture was then diluted with water and ethyl acetate. The aqueous phase was extracted with ethyl acetate, the combined organic phases were washed and dried and the solvent was removed. Column chromatography (petroleum ether/MTBE 8:1→3:1) gave 0.15 g (0.36 mmol, 61%) of the title compound.

1H-NMR (400 MHz, CDCl₃): δ=6.7 (s, 1H), 7.3 (s, 1H), 7.4 (s, 1H), 7.6 (t, 1H), 8.0 (s, 1H).

In accordance with process A:

2-Chloro-4-methoxy-6-(3-trifluoromethyl-1H-pyrazolyl)pyridine

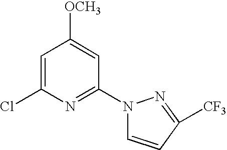

A mixture of 1 g (5.6 mmol) of 2,6-dichloro-4-methoxypyridine, 0.72 g (5.3 mmol) of 3-trifluoromethylpyrazole, 3.7 g (11 mmol) of cesium carbonate, 2 spatula tips of bis[copper(I) trifluoromethanesulfonate]benzene complex and 3 drops of ethyl acetate in xylene was stirred at 120° C. for 23 h. Following dilution with water, the mixture was extracted with ethyl acetate. The combined organic phases were washed, dried and freed from the solvent. This gave, after column chromatography (petroleum ether/ethyl acetate 100:0→0:100), 0.9 g (3.2 mmol, 61%) of the title compound.

4-Methoxy-6-(3-trifluoromethyl-1H-pyrazol-1-yl)-2-(5-trifluoro-methyl-3-thienyloxy)pyridine

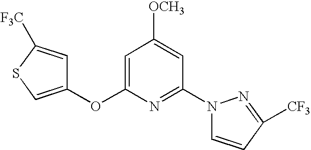

A mixture of 182 mg (1.1 mmol) of 3-hydroxy-5-trifluoromethylthiophene, 299 mg (2.2 mmol) of potassium carbonate, 1 spatula tip of 18-crown-6 and 300 mg (1.1 mmol) of 2-chloro-4-methoxy-6-(3-trifluoromethyl-1H-pyrazolyl)pyridine was stirred at 120° C. in 8 ml of N-methylpyrrolidone (NMP) for 5.5 h. MTBE/water 1:1 was added to the mixture, which was then extracted with methyl tert-butyl ether (MTBE). The combined organic phases were washed, dried and freed from the solvent. Chromatography gave 200 mg (0.5 mmol, 44%) of the title compound.

In accordance with process B:

2-Chloro-4-cyano-6-methylthiopyridine

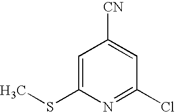

1.04 g (6 mmol) of 2,6-dichloro-4-cyanopyridine and 0.42 g (6 mmol) of sodium thiomethoxide were stirred in THF under reflux for 13 h. The solvent was removed under reduced pressure and the residue was then taken up in water and extracted with ethyl acetate. The combined organic phases were dried, concentrated and purified by column chromatography (cyclohexane/ethyl acetate 7:1→2:1), which gave 0.67 g (3.64 mmol, 61%) of the title compound.

4-Cyano-2-methylthio-6-(3-trifluoromethyl-1H-pyrazol-1-yl)pyridine

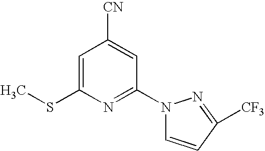

A mixture of 0.65 g (3.52 mmol) of 2-chloro-4-cyano-6-methylthiopyridine, 0.43 g (3.17 mmol) of 3-trifluoromethylpyrazole and 0.75 g (5.28 mmol) of potassium carbonate in DMF was stirred at 50° C. for 7 h and then at room temperature for 72 h. The reaction mixture was diluted with water and extracted with ethyl acetate. The combined organic phases were dried and freed from the solvent, which gave 0.59 g (2.08 mmol, 55%) of the title compound.

4-Cyano-2-methylsulfonyl-6-(3-trifluoromethyl-1H-pyrazol-1-yl)pyridine

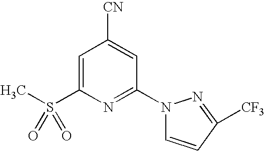

At 0-5° C., 1.82 g (2.96 mmol) of Oxone® in water were added dropwise to 560 mg (1.97 mmol) of 2-cyano-2-methylthio-6-(3-trifluoromethyl-1H-pyrazol-1-yl)pyridine in methanol. The pH of the solution was maintained at 2-3. The reaction mixture was stirred at room temperature for 14 h, diluted with water and extracted with ethyl acetate. The combined organic phases were washed, dried and freed from the solvent. This gave 540 mg (1.71 mmol, 87%) of the title compound.

4-Cyano-6-(3-trifluoromethyl-1H-pyrazol-1-yl)-2-(5-trifluoro-methyl-3-thienyloxy)pyridine

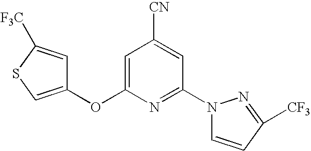

A mixture of 270 mg (1.62 mmol) of 3-hydroxy-5-trifluoromethylthiophene, 540 mg (1.71 mmol) of 4-cyano-2-methylsulfonyl-6-(3-trifluoromethyl-1H-pyrazol-1-yl)pyridine and 350 mg (2.57 mmol) of potassium carbonate in DMF was stirred at 80° C. for 7 h and then at room temperature for 72 h. The mixture was concentrated, water was added and the mixture was extracted with ethyl acetate. The combined organic phases were then dried and freed from the solvent. Column chromatography (petroleum ether/ethyl acetate 100:0→0:100) gave 500 mg (1.24 mmol, 76%) of the title compound.

In accordance with process D:

5-Methyl-2-(3-trifluoromethyl-1H-pyrazol-1-yl)pyridine

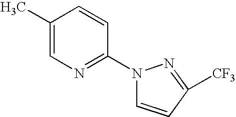

A mixture of 3.03 g (17.6 mmol) of 2-bromo-5-methylpyridine, 3.59 g (26.4 mmol) of 3-trifluoromethylpyrazole, 6.3 g (19.3 mmol) of cesium carbonate, 3.17 g (17.6 mmol) of phenanthroline, 2.06 g (8.8 mmol) of dibenzylideneacetone and a spatula tip of bis[copper(I) trifluoromethanesulfonate]benzene complex in xylene was stirred at 125° C. for 8 h and then at room temperature for 12 h. The reaction mixture was diluted with diethyl ether and the organic phase was washed with saturated ammonium chloride solution and with saturated sodium chloride solution. After drying and removal of the solvent, the reaction mixture was purified by column chromatography (petroleum ether/MTBE 100:0→50:50), which gave 3.0 g (13.2 mmol, 75%) of the title compound.

5-Methyl-2-(3-trifluoromethyl-1H-pyrazol-1-yl)pyridine N-oxide

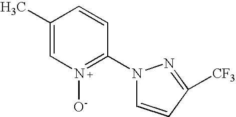

2 spatula tips of sodium tungstate and a total of 11.9 ml of a 30% strength solution of hydrogen peroxide were added to 2.6 g (11 mmol) of 5-methyl-2-(3-trifluoromethyl-1H-pyrazol-1-yl)pyridine in trifluoroacetic acid. After 96 h at room temperature, the reaction mixture was diluted with water and extracted with ethyl acetate. The combined organic phases were then washed, dried and freed from the solvent. Column chromatography (cyclohexane/ethyl acetate 95:5→0:100) gave 1.6 g (6.6 mmol, 60%) of the title compound.

2-Chloro-3-methyl-6-(3-trifluoromethyl-1H-pyrazol-1-yl)pyridine

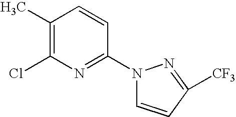

At 80° C., 1.7 g (7 mmol) of 5-methyl-2-(3-trifluoromethyl-1H-pyrazol-1-yl)pyridine N-oxide were added a little at a time to 2.15 g (14 mmol) of phosphorus oxytrichloride, and the mixture was stirred at this temperature for 4 h. With cooling, the reaction mixture was hydrolyzed and extracted with ethyl acetate. The combined organic phases were then washed, dried and freed from the solvent. Column chromatography (cyclohexane/ethyl acetate 100:9→95:5) gave 1.1 g (4.2 mmol, 60%) of the title compound.

3-Methyl-6-(3-trifluoromethyl-1H-pyrazol-1-yl)-2-(5-trifluoromethyl-3-thienyloxy)pyridine

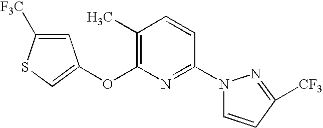

A mixture of 0.38 g (2.3 mmol) of 3-hydroxy-5-trifluoromethylthiophene, 0.5 g (1.9 mmol) of 2-chloro-3-methyl-6-(3-trifluoromethyl-1H-pyrazol-1-yl)pyridine, 0.14 g (1 mmol) of copper(I) bromide and 0.52 g (3.8 mmol) of potassium carbonate in DMF was stirred at 120° C. for 15 h and at room temperature for 60 h. The mixture was concentrated, water was added and the mixture was extracted with ethyl acetate. The combined organic phases were then washed, dried and freed from the solvent. Chromatography (silica gel RP-18, methanol/water 8:2→9:1) gave 0.15 g (0.4 mmol, 20%) of the title compound.

In addition to the above compounds, Tables 2 and 3 list further pyrazolyl-substituted thienyloxypyridines of the formula I and thienyloxypyridines of the formula XIII which were prepared or are preparable in an analogous manner by the processes described above.

In addition to the above compounds, Table 4 lists further 3-trifluoromethyl-1H-pyrazol-1-yl-substituted pyridines of the formula III which were prepared or are preparable in an analogous manner by the processes described above.

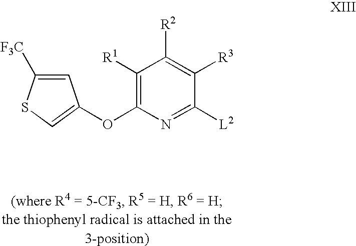

TABLE 2

| No. | R¹ | R² | R³ | L² | ¹H-NMR [400 MHz, CDCl₃] |
|---|---|---|---|---|---|
| 2.1 | F | H | F | F | 7.3 (s, 1H), 7.35 (s, 1H), 7.5 |
| | | | | | (m, 1H) |

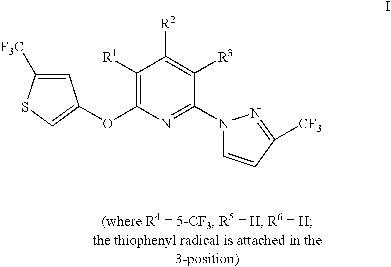 TABLE 3

| No. | R¹ | R² | R³ | ¹H-NMR (400 MHz, CDCl₃) |
|---|---|---|---|---|
| 3.1 | H | H | H | 6.7 (s, 1H), 6.9 (d, 1H), 7.2 |
| | | | | (s, 1H), 7.4 (s, 1H), 7.8 |
| | | | | (d, 1H), 7.9 (t, 1H), 8.2 (s, 1H) |
| 3.2 | H | CN | H | 6.7 (s, 1H), 7.1 (s, 1H), 7.3 |
| | | | | (s, 1H), 8.0 (s, 1H), 8.2 (s, 1H) |
| 3.3 | H | OCH₃ | H | 4.0 (s, 1H), 6.3 (s, 1H), 6.6 |
| | | | | (s, 1H), 7.2 (s, 1H), 7.3 |
| | | | | (s, 1H), 7.4 (s, 1H), 8.2 (s, 1H) |
| 3.4 | CN | H | H | 6.7 (s, 1H), 7.3 (s, 1H), 7.4 |
| | | | | (s, 1H), 7.8 (d, 1H), 8.1 |
| | | | | (s, 1H), 8.2 (d, 1H) |
| 3.5 | CF₃ | H | H | 6.7 (s, 1H) , 7.3 (s, 1H), 7.4 |
| | | | | (s, 1H), 7.8 (d, 1H), 8.1 |
| | | | | (s, 1H); 8.2 (d, 1H) |
| 3.6 | F | H | F | 6.7 (s, 1H), 7.3 (s, 1H), 7.4 |
| | | | | (s, 1H), 7.6 (t, 1H), 8.0 (s, 1H) |
| 3.7 | Cl | Cl | Cl | 6.8 (s, 1H), 7.1 (s, 1H), 7.2 |
| | | | | (s, 1H), 7.7 (s, 1H) |
| 3.8 | CH₃ | H | H | 2.4 (s, 3H), 6.6 (d, 1H), 7.2 |
| | | | | (s, 1H), 7.4 (s, 1H), 7.6 |
| | | | | (d, 1H), 7.7 (d, 1H), 8.1 (d, 1H) |

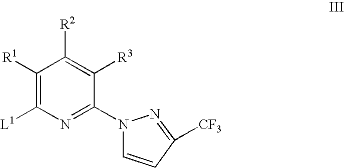 TABLE 4

| No. | R¹ | R² | R³ | L¹ | ¹H-NMR (400 MHz, CDCl₃ |
|---|---|---|---|---|---|
| 4.1 | H | OCH₃ | H | Cl | 4.0 (s, 3H), 6.7 (s, 1H), |
| | | | | | 6.8 (s, 1H), 7.5 (s, 1H), |
| | | | | | 8.6 (s, 1H) |
| 4.2 | H | CN | H | SO₂CH₃ | 3.3 (s, 3H), 6.8 (s, 1H) |
| | | | | | 8.2 (s, 1H), 8.5 (s, 1H), |
| | | | | | 8.6 (s, 1H) |
| 4.3 | CH₃ | H | H | Cl | 6.7 (s, 1H), 7.7 (d, 1H), |
| | | | | | 7.9 (d, 1H), 8.6 (s, 1H) |
| 4.4 | H | H | H | SO₂CH₃ | 3.2 (s, 3H), 6.8 (s, 1H), |
| | | | | | 8.0 (d, 1H), 8.1 (t. 1H), |
| | | | | | 8.3 (d, 1H), 8.6 (s, 1H) |
| 4.5 | CN | H | H | Cl | 6.8 (s, 1H), 8.1 (d, 1H), |
| | | | | | 8.1 (d, 1H), 8.6 (s, 1H) |
| 4.6 | CF₃ | H | H | Cl | 6.7 (s, 1H), 8.1 (d, 1H), |
| | | | | | 8.2 (d, 1H), 8.6 (s, 1H) |
| 4.7 | Cl | Cl | Cl | Cl | 6.5 (s, 1H), 7.2 (s, 1H) |

Use

The pyrazolyl-substituted thienyloxypyridines of the formula I and their agriculturally useful salts are suitable, both in the form of isomer mixtures and in the form of the pure isomers, as herbicides. The herbicidal compositions comprising compounds of the formula I control vegetation on non-crop areas very efficiently, especially at high rates of application. They act against broad-leaved weeds and harmful grasses in crops such as wheat, rice, maize, soya and cotton without causing any significant damage to the crop plants. This effect is mainly observed at low rates of application.

Depending on the application method used, the compounds of the formula I or the herbicidal compositions comprising them can additionally be employed in a further number of crop plants for eliminating undesirable plants. Examples of suitable crops are the following:

Allium cepa, Ananas comosus, Arachis hypogaea, Asparagus officinalis, Beta vulgaris spec. altissima, Beta vulgaris spec. rapa, Brassica napus var. napus, Brassica napus var. napobrassica, Brassica rapa var. silvestris, Camellia sinensis, Carthamus tinctorius, Carya illinoinensis, Citrus limon, Citrus sinensis, Coffea arabica (Coffea canephora, Coffea liberica), Cucumis sativus, Cynodon dactylon, Daucus carota, Elaeis guineensis, Fragaria vesca, Glycine max, Gossypium hirsutum, (Gossypium arboreum, Gossypium herbaceum, Gossypium vitifolium), Helianthus annuus, Hevea brasiliensis, Hordeum vulgare, Humulus lupulus, Ipomoea batatas, Juglans regia, Lens culinaris, Linum usitatissimum, Lycopersicon lycopersicum, Malus spec., Manihot esculenta, Medicago sativa, Musa spec., Nicotiana tabacum (N. rustica), Olea europaea, Oryza sativa, Phaseolus lunatus, Phaseolus vulgaris, Picea abies, Pinus spec., Pisum sativum, Prunus avium, Prunus persica, Pyrus communis, Ribes sylvestre, Ricinus communis, Saccharum officinarum, Secale cereale, Solanum tuberosum, Sorghum bicolor (s. vulgare), Theobroma cacao, Trifolium pratense, Triticum aestivum, Triticum durum, Vicia faba, Vitis vinifera and Zea mays.

In addition, the compounds of the formula I may also be used in crops which tolerate the action of herbicides owing to breeding, including genetic engineering methods.

The compounds of the formula I, or the herbicidal compositions comprising them, can be used for example in the form of ready-to-spray aqueous solutions, powders, suspensions, also highly-concentrated aqueous, oily or other suspensions or dispersions, emulsions, oil dispersions, pastes, dusts, materials for broadcasting or granules, by means of spraying, atomizing, dusting, broadcasting or watering. The use forms depend on the intended aims; in any case, they should ensure a very fine distribution of the active compounds according to the invention.

The herbicidal compositions comprise a herbicidally effective amount of at least one compound of the formula I or an agriculturally useful salt of I and auxiliaries customary for formulating crop protection agents.

Essentially, suitable inert auxiliaries include:

mineral oil fractions of medium to high boiling point, such as kerosene and diesel oil, furthermore coal tar oils and oils of vegetable or animal origin, aliphatic, cyclic and aromatic hydrocarbons, e.g. paraffins, tetrahydronaphthalene, alkylated naphthalenes and their derivatives, alkylated benzenes and their derivatives, alcohols such as methanol, ethanol, propanol, butanol and cyclohexanol, ketones such as cyclohexanone, strongly polar solvents, e.g. amines such as N-methylpyrrolidone, and water.

Aqueous use forms can be prepared from emulsion concentrates, suspensions, pastes, wettable powders or water-dispersible granules by adding water. To prepare emulsions, pastes or oil dispersions, the substrates, either as such or dissolved in an oil or solvent, can be homogenized in water by means of a wetting agent, tackifier, dispersant or emulsifier. Alternatively, it is also possible to prepare concentrates consisting of active substance, wetting agent, tackifier, dispersant or emulsifier and, if desired, solvent or oil, which are suitable for dilution with water.

Suitable surfactants (adjuvants) are the alkali metal salts, alkaline earth metal salts and ammonium salts of aromatic sulfonic acids, e.g. ligno-, phenol-, naphthalene- and dibutylnaphthalenesulfonic acid, and of fatty acids, alkyl- and alkylarylsulfonates, alkyl sulfates, lauryl ether sulfates and fatty alcohol sulfates, and salts of sulfated hexa-, hepta- and octadecanols, and also of fatty alcohol glycol ethers, condensates of sulfonated naphthalene and its derivatives with formaldehyde, condensates of naphthalene, or of the naphthalenesulfonic acids with phenol and formaldehyde, polyoxyethylene octylphenol ether, ethoxylated isooctyl-, octyl- or nonylphenol, alkylphenyl polyglycol ether or tributylphenyl polyglycol ether, alkylaryl polyether alcohols, isotridecyl alcohol, fatty alcohol ethylene oxide condensates, ethoxylated castor oil, polyoxyethylene alkyl ethers or polyoxypropylene alkyl ethers, lauryl alcohol polyglycol ether acetate, sorbitol esters, lignosulfite waste liquors or methylcellulose.

Powders, materials for broadcasting and dusts can be prepared by mixing or grinding the active substances together with a solid carrier.

Granules, e.g. coated granules, impregnated granules and homogeneous granules, can be prepared by binding the active compounds to solid carriers. Solid carriers are mineral earths, such as silicas, silica gels, silicates, talc, kaolin, limestone, lime, chalk, bole, loess, clay, dolomite, diatomaceous earth, calcium sulfate, magnesium sulfate, magnesium oxide, ground synthetic materials, fertilizers such as ammonium sulfate, ammonium phosphate, ammonium nitrate and ureas, and products of vegetable origin, such as cereal meal, tree bark meal, wood meal and nutshell meal, cellulose powders, or other solid carriers.

The concentrations of the compounds of the formula I in the ready-to-use preparations can be varied within wide ranges. In general, the formulations comprise from about 0.001 to 98% by weight, preferably 0.01 to 95% by weight of at least one active compound. The active compounds are employed in a purity of from 90% to 100%, preferably from 95% to 100% (according to the NMR spectrum).

The production of such preparations is illustrated by the following formulation examples:

I. 20 parts by weight of an active compound of the formula I are dissolved in a mixture consisting of 80 parts by weight of alkylated benzene, 10 parts by weight of the adduct of 8 to 10 mol of ethylene oxide to 1 mol of oleic acid N-monoethanolamide, 5 parts by weight of calcium dodecylbenzenesulfonate and 5 parts by weight of the adduct of 40 mol of ethylene oxide to 1 mol of castor oil. Pouring the solution into 100,000 parts by weight of water and finely distributing it therein gives an aqueous dispersion which comprises 0.02% by weight of the active compound.

II. 20 parts by weight of an active compound of the formula I are dissolved in a mixture consisting of 40 parts by weight of cyclohexanone, 30 parts by weight of isobutanol, 20 parts by weight of the adduct of 7 mol of ethylene oxide to 1 mol of isooctylphenol and 10 parts by weight of the adduct of 40 mol of ethylene oxide to 1 mol of castor oil. Pouring the solution into 100,000 parts by weight of water and finely distributing it therein gives an aqueous dispersion which comprises 0.02% by weight of the active compound.

III. 20 parts by weight of an active compound of the formula I are dissolved in a mixture consisting of 25 parts by weight of cyclohexanone, 65 parts by weight of a mineral oil fraction of boiling point 210 to 280° C. and 10 parts by weight of the adduct of 40 mol of ethylene oxide to 1 mol of castor oil. Pouring the solution into 100,000 parts by weight of water and finely distributing it therein gives an aqueous dispersion which comprises 0.02% by weight of the active compound.

IV. 20 parts by weight of an active compound of the formula I are mixed thoroughly with 3 parts by weight of sodium diisobutylnaphthalenesulfonate, 17 parts by weight of the sodium salt of lignosulfonic acid from a sulfite waste liquor and 60 parts by weight of pulverulent silica gel, and the mixture is ground in a hammer mill. Finely distributing the mixture in 20,000 parts by weight of water gives a spray mixture which comprises 0.1% by weight of the active compound.

V. 3 parts by weight of an active compound of the formula I are mixed with 97 parts by weight of finely divided kaolin. This gives a dust which comprises 3% by weight of the active compound.

VI. 20 parts by weight of an active compound of the formula I are mixed intimately with 2 parts by weight of calcium dodecylbenzenesulfonate, 8 parts by weight of fatty alcohol polyglycol ether, 2 parts by weight of the sodium salt of a phenol/urea/formaldehyde condensate and 68 parts by weight of a paraffinic mineral oil. This gives a stable oily dispersion.

VII. 1 part by weight of an active compound of the formula I is dissolved in a mixture consisting of 70 parts by weight of cyclohexanone, 20 parts by weight of ethoxylated isooctylphenol and 10 parts by weight of ethoxylated castor oil. This gives a stable emulsion concentrate.

VIII. 1 part by weight of an active compound of the formula I is dissolved in a mixture of 80 parts by weight of cyclohexanone and 20 parts by weight of Wettol^{R} EM 31

(=nonionic emulsifier based on ethoxylated castor oil).

This gives a stable emulsion concentrate.

The compounds of the formula I or the herbicidal compositions can be applied pre- or post-emergence. If the active compounds are less well tolerated by certain crop plants, application techniques may be used in which the herbicidal compositions are sprayed, with the aid of the spraying equipment, in such a way that they come into contact as little as possible, if at all, with the leaves of the sensitive crop plants, while the active compounds reach the leaves of undesirable plants growing underneath, or the bare soil surface (post-directed, lay-by).

The application rates of the compound of the formula I are from 0.001 to 3.0, preferably from 0.01 to 1.0 kg/ha of active substance (a.s.), depending on the control target, the season, the target plants and the growth stage.

To widen the activity spectrum and to achieve synergistic effects, the pyrazolyl-substituted thienyloxypyridines of the formula I may be mixed with a large number of representatives of other herbicidal or growth-regulating active compound groups and then applied concomitantly. Suitable components for mixtures are, for example, 1,2,4-thiadiazoles, 1,3,4-thiadiazoles, amides, aminophosphoric acid and its derivatives, aminotriazoles, anilides, (hetero)aryloxyalkanoic acids and their derivatives, benzoic acid and its derivatives, benzothiadiazinones, 2-(hetaroyl/aroyl)-1,3-cyclohexanediones, heteroarylaryl ketones, benzylisoxazolidinones, meta-CF₃-phenyl derivatives, carbamates, quinolinecarboxylic acid and its derivatives, chloroacetanilides, cyclohexenone oxime ether derivatives, diazines, dichloropropionic acid and its derivatives, dihydrobenzofurans, dihydrofuran-3-ones, dinitroanilines, dinitrophenols, diphenyl ether, dipyridyls, halocarboxylic acids and their derivatives, ureas, 3-phenyluracils, imidazoles, imidazolinones, N-phenyl-3,4,5,6-tetrahydrophthalimides, oxadiazoles, oxiranes, phenols, aryloxy- and heteroaryloxyphenoxypropionic esters, phenylacetic acid and its derivatives, 2-phenylpropionic acid and its derivatives, pyrazoles, phenylpyrazoles, pyridazines, pyridinecarboxylic acid and its derivatives, pyrimidyl ethers, sulfonamides, sulfonylureas, triazines, triazinones, triazolinones, triazolecarboxamides and uracils.

It may furthermore be advantageous to apply the compounds of the formula I, alone or else concomitantly in combination with other herbicides, or in the form of a mixture with other crop protection agents, for example together with agents for controlling pests or phytopathogenic fungi or bacteria. Also of interest is the miscibility with mineral salt solutions, which are employed for treating nutritional and trace element deficiencies. Non-phytotoxic oils and oil concentrates may also be added.

We claim:

1. A pyrazolyl-substituted thienyloxypyridine of the formula I 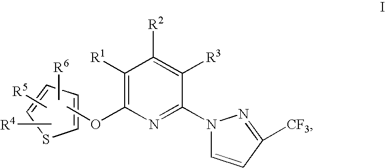
where
R¹, R³ are hydrogen, halogen, cyano, nitro, C₁-C₆-alkyl, C₁-C₆-haloalkyl, C₁-C₆-alkoxy or C₁-C₆-haloalkoxy;
R² is hydrogen, halogen, cyano, C₂-C₆-alkenyl, C₂-C₆-alkynyl, C₁-C₆-haloalkyl, C₂-C₆-haloalkenyl, C₂-C₆-haloalkynyl, C₁-C₆-alkoxy, C₃-C₆-alkenyloxy, C₃-C₆-alkynyloxy, C₁-C₆-haloalkoxy, C₁-C₆-alkoxy-C₁-C₄-alkyl, C₁-C₆-alkylamino, di(C₁-C₄-alkyl)amino, C₁-C₆-alkylthio, C₁-C₆-haloalkylthio, C₁-C₆-alkylsulfinyl, C₁-C₆-haloalkylsulfinyl, C₁-C₆-alkylsulfonyl, C₁-C₆-haloalkylsulfonyl or COR⁷;
R⁴, R⁵, R⁶ are hydrogen, halogen, cyano, C₁-C₆-alkyl, C₁-C₆-haloalkyl, C₁-C₆-alkoxy, C₁-C₆-haloalkoxy, C₁-C₆-alkylthio, C₁-C₆-haloalkylthio, C₁-C₆-alkylsulfonyl or C₁-C₆-haloalkylsulfonyl;
R⁷ is hydrogen, hydroxyl, C₁-C₆-alkyl, C₁-C₆-alkoxy, amino, C₁-C₆-alkylamino or di(C₁-C₄-alkyl)amino;
and its agriculturally useful salts.

2. A pyrazolyl-substituted thienyloxypyridine of the formula I as claimed in claim 1 where
R¹, R³ are hydrogen, halogen, cyano, nitro, C₁-C₆-alkyl or C₁-C₆-haloalkyl;
R² is hydrogen, halogen, cyano, C₁-C₆-haloalkyl, C₁-C₆-alkoxy, C₁-C₆-alkylamino, di(C₁-C₄-alkyl)amino, C₁-C₆-alkylthio or COR⁷.

3. A pyrazolyl-substituted thienyloxypyridine of the formula I as claimed in claim 1 or 2 where
R⁴, R⁵, R⁶ are hydrogen, halogen, cyano, C₁-C₆-alkyl, C₁-C₆-haloalkyl or C₁-C₆-haloalkoxy.

4. A process for preparing a pyrazolyl-substituted thienyloxypyridine of the formula I as claimed in claim 1, which comprises reacting 3-trifluoromethyl-1H-pyrazol-1-yl-substituted pyridines of the formula III 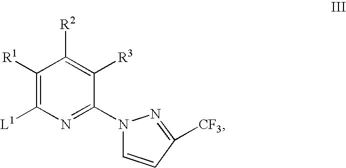
where R¹, R² and R³ are as defined in claim 1 and L¹ is a nucleophilically displaceable leaving group,
with a hydroxythiophene of the formula II 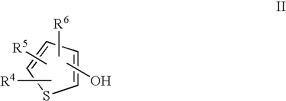
where R⁴, R⁵ and R⁶ are as defined in claim 1.

5. A process for preparing pyrazolyl-substituted thienyloxypyridines of the formula I as claimed in claim 1, which comprises reacting thienyloxypyridine derivatives of the formula XIII 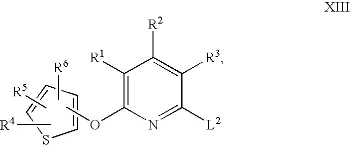
where R¹, R², R³, R⁴, R⁵ and R⁶ are as defined in claim 1 and L² is a nucleophilically displaceable leaving group,
with a pyrazole derivative of the formula IV

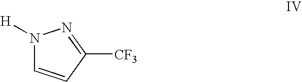

6. A 3-trifluoromethyl-1H-pyrazol-1-yl-substituted pyridine of the formula III 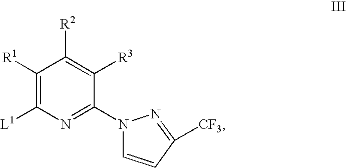
where R¹, R² and R³ are as defined in claim 1 and L¹ is a nucleophilically displaceable leaving group.

7. A thienyloxypyridine derivative of the formula XIII 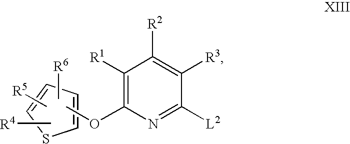
where R¹, R², R³, R⁴, R⁵ and R⁶ are as defined in claim 1 and L² is a nucleophilically displaceable leaving group.

8. A composition, comprising a herbicidally effective amount of at least one pyrazolyl-substituted thienyloxypyridine of the formula I or of an agriculturally useful salt of I as claimed in any of claims 1 to 3 and auxiliaries customary for formulating crop protection agents.

9. A process for preparing compositions as claimed in claim 8, which comprises mixing a herbicidally effective amount of at least one pyrazolyl-substituted thienyloxypyridine derivative of the formula I or of an agriculturally useful salt of I as claimed in any of claims 1 to 3 and auxiliaries customary for formulating crop protection agents.

10. A method for controlling undesirable vegetation, which comprises allowing a herbicidally effective amount of at least one pyrazolyl-substituted thienyloxypyridine derivative of the formula I or of an agriculturally useful salt of I as claimed in any of claims 1 to 3 to act on plants, their habitat and/or on seeds.

11. The use of the pyrazolyl-substituted thienyloxypyridine derivatives of the formula I and their agriculturally useful salts as claimed in any of claims 1 to 3 as herbicides.
